(19) United States
(12) Patent Application Publication
(10) Pub. No.: US 2024/0172756 A1
(43) Pub. Date: May 30, 2024

(54) ORGANOANTIMONY(V) CYANOXIMATE COMPOUNDS AND METHODS OF PRODUCTION AND USE THEREOF

(71) Applicants: Board of Regents for Oklahoma Agricultural and Mechanical Colleges, Stillwater, OK (US); Missouri State University, Springfield, MO (US)

(72) Inventors: Nikolay Gerasimchuk; Kevin PINKS, Springfield, MO (US); Marianna PATRAUCHAN, Stillwater, OK (US); Karen WOZNIAK, Stillwater, OK (US)

(21) Appl. No.: 18/547,610

(22) PCT Filed: Feb. 23, 2022
(86) PCT No.: PCT/US22/17465

§ 371 Date: Aug. 23, 2023

Related U.S. Application Data
(60) Provisional application No. 63/152,490, filed on Feb. 23, 2021.

(51) Int. Cl. A01N 55/02; A01P 1/00; A01P 3/00; A61K 31/555; A61P 31/04; A61P 31/10; C07F 9/92; C09D 5/14; C09D 7/63; C09J 9/00; C09J 11/06

(52) U.S. Cl. CPC A01N 55/02; A01P 1/00; A01P 3/00; A61K 31/555; A61P 31/04; A61P 31/10; C07F 9/92; C09D 5/14; C09D 7/63; C09J 9/00; C09J 11/06

(57) ABSTRACT

Organoantimony(V) cyanoximate compounds are disclosed, as well as compositions containing same. Also disclosed are methods of producing and using the organoantimony(V) cyanoximate compounds and compositions.

### CROSS REFERENCE TO RELATED APPLICATIONS/INCORPORATION BY REFERENCE STATEMENT

This application claims benefit under 35 USC § 119(e) of U.S. Provisional Application No. 63/152,490, filed Feb. 23, 2021. The entire contents of the above-referenced application(s) are hereby expressly incorporated herein by reference.

### STATEMENT REGARDING FEDERALLY SPONSORED RESEARCH OR DEVELOPMENT

Not Applicable.

### BACKGROUND

Microbial infections are associated with considerable morbidity and costs. The continuous spread of antimicrobial resistance (AMR) among bacterial and fungal pathogens imperils the usefulness of antibiotics and antifungals, which previously revolutionized and enabled medical interventions. The increasing AMR is manifesting as multidrug resistance (MDR) leading to a global crisis that if not addressed will soon have a huge adversarial impact on human race, The main causes of the rapid increase of AMR include an overuse of antimicrobials, inappropriate prescribing, and the inability of patients to follow the prescribed drug regimen. Extensive use of antibiotics in agricultural settings, as well as wide availability of some antibiotics are also important factors. This situation led to the appearance of antibiotic resistant bacterial pathogens, exemplified by Methicillin-Resistant Staphylococcus aureus (MRSA), and Vancomycin-Resistant Enterococci (VRE), which are becoming increasingly difficult to treat with the current range of available antibiotics. Fungal pathogens Cryptococcus neoformans and Candida albicans are also becoming increasingly resistant to antifungal drugs, and different Candida species with natural drug resistance, such as Candida auris, are evolving in hospital settings and becoming major pathogens. The MDR phenomenon is one of the greatest global public health challenges that humanity is currently facing. According to the Center for Disease Control (CDC) Prevention's report on antimicrobial resistance conducted in 2019, annually, there are more than 2.8 million antibiotic-resistant bacterial infections, from which more than 35,000 people die in the US. Although fungal drug-resistant strains are more difficult to track, current estimates indicate that fungal infections lead to more than 1.5 million deaths annually worldwide. Aside from that, the economic impacts due to treating these infections as well as from the loss of productivity is astronomical—about $4.6 billion annually for bacterial infections and about $7.2 billion annually for fungal infections.

With modern medicine's reliance on antimicrobials in treating diseases such as pneumonia, .tuberculosis, sexually transmitted diseases, and bloodstream infections, .the problem with AMR is that soon no current treatments will be effective. Therefore, the development of new antimicrobial agents is imperative. The critically important progress in this field requires the development of new non-antibiotic chemical compounds that would inhibit bacterial and fungal growth, but at the same time, would not be toxic to human tissues. Pure organic compounds cannot meet these criteria, as they are rather quickly metabolized by microorganisms, while simple inorganic salts and Werner-type complexes are toxic or not soluble.

The successful application of metal complexes and organometallic compounds in treatments of numerous human diseases is a vigorously expanding area in both biomedical and bioinorganic chemistry research. Remarkably, the group V elements in the Periodic Table—pnictogens—contains all biologically active elements. Nitrogen (N) and phosphorous (P) are crucial for life on the planet, while arsenic (As), antimony (Sb), and bismuth (Bi) possess very useful properties for biomedical applications. Paul Ehrlich, widely known as the father of antimicrobial chemotherapy and bioinorganic chemistry, found the “magic bullet” (compound 606) capable of treating the highly infectious bacterial pathogen Treponema pallidum without hurting the host, This compound is a heterocyclic arsenic-based small molecule (C₁₂H₁₃As₂ClN₂O₂) that was used to successfully treat syphilis and was marketed as Salvarsan (“Lifesaving”). Despite the well-known toxicity of arsenic, this drug saved >560,000 lives within fifty years. Bismuth, contrary to arsenic, is not toxic and is widely used as subsalicylate (C₇H₅BiO₄) in the well-known drug Pepto-Bismol. In turn, antimony has been used since early Egyptian's civilization, For example, NaSbO₃ was commonly used as an emetic compound until the late 1700's. The other successful application of Sb is the treatment of leishmaniasis, caused by a protozoan parasite Leishmania transmitted through the bite of infected sandflies in South and Central America, Bangladesh, southern Europe, and North Africa. The active compounds against the disease were found to be several Sb(V) carbohydrates, such as sodium antimony gluconate (Pentostam) and megiumine antimonate (Glucantime). These have been in use for more than six decades to treat leishmaniasis. Moreover, several organoantimony compounds have been studied and shown to possess antimicrobial, antifungal, and antitumor activities (Gasser et al., J Med Chem (2011) 54(1):3-25; Frezard et al., Molecules (2009) 14(7):2317-36); Agrawal et al., J Coord Chem (2011) 64(3):554-563; Khan et al., Middle Fast Journal of Scientific Research (2013) 17:705.-711; Oliveira et al., Molecules (2011) 16(12):10314-23; Sharma et al., Bioinorg Chem Appl (2006) 16895; and Singh et al., Main Group Metal Chemistry (2010) 33).

Recently, it was discovered that Sb(V) cyanoximates are thermally and chemically stable both in solid state and solutions (Domasevitch et al., Inorg Chem (2000) 39(6):1227-1237). In addition, no intrinsic in vitro cytotoxicity was detected for free cyanoximes, organic ligands used to synthesize organoantimony(V) (Eddings et al., Inorg Chem (2004) 43(13):3894-909; Gerasimchuk et al., Inorg Chem (2007) 46(18):7268-84.); and Mann et al., Inorganica Chimica Acta (2016) 440:118-128).

Therefore, there is a need in the art for new and improved antimicrobial organometallic compounds and compositions that efficiently inhibit pathogenic bacteria and fungi. It is to such new and improved compounds and compositions, as well as methods of producing and using same, that the present disclosure is directed.

### BRIEF DESCRIPTION OF THE DRAWINGS

FIG. 1 includes a schematic representation of a metathesis reaction used for preparation of organoantimony(V) cyanoximate compounds in accordance with the present disclosure. In particular (but not by way of limitation), this figure demonstrates the preparation of tetraphenyl-antimony(V) compounds with selected cyanoximes via their Ag(I) derivatives. The rationale for choosing a specific cyanoxime compounds is provided together with their commonly used abbreviations. The most antimicrobially active cyanoximes in this series of G1 compounds are HACO and HMCO.

FIG. 2 includes a schematic representation of preparation of trimethylantimony(V) compounds with selected cyanoximes (and their abbreviations). The rationale for choosing this series of chlorinated cyanoximes is based on their strong biological activity. Compounds of this G2 series that showed pronounced antimicrobial activity are 2,6-diCl-PhCO and MCO.

FIG. 3 includes crystal structures of various tetraphenylantimony(V) cyanoximate compounds constructed in accordance with the present disclosure. A—SbPh₄(3PCO); B—SbPh₄(2PCO); C—SbPh₄(4PCO); D—SbPh₄(ECO) (showing two crystallographically independent molecules in the asymmetric unit); E—SbPh₄(MCO) (showing two crystallographically independent molecules in the asymmetric unit); F—for SbPh₄(TCO) (showing superimposed two co-crystallized diastereorners of the anion in the same asymmetric unit); and G—SbPh₄(TDCO) (showing two co-crystallized diastereomers syn- and anti- of the anion in the same asymmetric unit; important C—H—O intramolecular interactions that stabilize particular diastereomer are shown in dotted lines). The H-atoms are omitted for clarity.

FIG. 4 includes crystal structures of various trimethylantimony(V) bis-cyanoximate compounds constructed in accordance with the present disclosure. A—SbMe₃(ACO)₂; B—SbMe₃(4Cl-PhCO)₂; C—SbMe₃(2,4diCl-PhCO)₂; D—SbMe₃(TDCO)₂; and E—SbMe₃(TCO)₂. The H-atoms in presented structures are omitted for clarity.

FIG. 5 includes an analysis of organoantimony(V) cyanoximate compounds constructed in accordance with the present disclosure. A, B—actual appearance as crystalline solid of G1 series SbPh₄(MCO) and powder of G2 series SbMe₃(TDCO)₂. C—traces of weight loss (green) and heat flow (blue) for SbPh₄(ECO) showing the melting point of the compound at 145° C. without decomposition.

FIG. 6 includes representative images showing clearance zones in disc diffusion assays.

FIG. 7. J774.1. macrophages (5×10⁵ cells/ml) were incubated in cell culture media alone or cell culture media with each antimicrobial compound at the highest MIC concentration for 24 hours at 37° C, 5% CO₂. Percent cytotoxicity was calculated as per manufacturer's instructions. Data shown are means ± standard error of the means (SEM) of the cumulative results of three independent experiments (n=3), with each condition performed in triplicate.

FIG. 8. A—Schematic representation of three non-limiting embodiments of types of organoantimony(V) cyanoximate compounds constructed in accordance with the present disclosure (labeled G1, G2, and G3). The number of cyanoxirne residues increases, potentiating enhanced efficacy of target compounds, while the number of organic groups decreases, aiming to decrease hydrophobicity of the compound. B—several halogenated and water soluble cyanoximes utilized in production of organoantimony(V) cyanoximate compounds in accordance with the present disclosure. Asterisks indicate ligands readily available in the Gerasimchuk lab ligands for making organometallics of G1-G3 types,

FIG. 9A provides the chemical name and structure for certain cyanoximate compounds utilized in accordance with the present disclosure.

FIG. 9B provides generalized chemical formulations for monoximes, dioximes, and trioximes of cyanoximes synthesized in accordance with the present disclosure.

FIG. 9C illustrates the chemical structures of various members of different classes of cyanoximes synthesized in accordance with the present disclosure. Asterisks indicate cyanoximes for which crystal structures have been determined.

FIG. 10 graphically demonstrates that certain non-limiting embodiments of organoantimony(V) compounds produced and tested in accordance with the present disclosure exhibited antifungal activity against C. neoformans and/or C. albicans.

FIG. 11 graphically demonstrates that the compound Sb(Ph)₄(MCO) exhibited antifungal activity against both C. neoformans as well as C. albicans, whereas none of the three known starting compounds possessed any antifungal activity.

FIG. 12 demonstrates a procedure for analyzing the antibacterial activity of organoantimony(V) cyanoximate compounds constructed in accordance with the present disclosure in paper disk studies, wherein the organoantimony(V) cyanoximate compounds are deposited onto the paper disks in solid state, powder form and tested against the pathogens P. aeruginosa, S. aureus, and E. coli.

FIGS. 13, 14, and 15 demonstrate paper disk clearance zone studies during growth on solid media for organoantimony(V) cyanoximate compounds constructed in accordance with the present disclosure. Organoantimony(V) cyanoximate compounds containing the cyanoximates ACO, ECO, or TCO (FIGS. 13, 14, and 15, respectively), were deposited in solid state, powder form on paper disks and tested against the pathogens P. aeruginosa, S. aureus, and E. coli, in the manner described in FIG. 12.

FIG. 16 demonstrates a procedure for analyzing the antibacterial activity of organoantimony(V) cyanoximate compounds constructed in accordance with the present disclosure in liquid cultures, wherein the organoantimony(V) cyanoximate compounds are solubilized in DMSO and tested in solution against the pathogens P. aeruginosa, S. aureus, and E. coli.

FIGS. 17 and 18 demonstrate MIC inhibition assays for various solubilized organoantimony(V) cyanoximate compounds constructed in accordance with the present disclosure against the pathogens P. aeruginosa, S. aureus, and E. coli, in the manner described in FIG. 16.

FIG. 19 graphically demonstrates antifungal activity of several organoantimony(V) compounds constructed in accordance with the present disclosure against C. neoformans and C. albicans.

FIG. 20 graphically demonstrates antibacterial activity of various organoantimony(V) compounds constructed in accordance with the present disclosure against Staphylococcus aureus MRSA in a broth dilution assay.

FIG. 21 graphically demonstrates antibacterial activity of various organoantimony(V) compounds constructed in accordance with the present disclosure against E. coli STEC in a broth dilution assay.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art, Standard techniques are used for chemical syntheses and chemical analyses,

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise, Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z, The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/ device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or, seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, SCA, ACS, BAC, or CAB. Continuing with this example, expressly included are combinations that contain repeats of one or more item or term, such as BB, AAA, AAB, BBC, AAABCCCC, CBBAAA, CABABB, and so forth, The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item,

The terms “analog,” “derivative,” or “variant” as used herein will be understood to refer to a variation of the normal or standard form or the wild-type form of molecules. For polypeptides, an analog may be a variant (polymorphism), a mutant, and/or a naturally or artificially chemically modified version of the wild-type polypeptide (including combinations of the above). Such analogs may have higher, full, intermediate, or lower activity than the normal form of the molecule, or no activity at all. Alternatively, and/or in addition thereto, for a chemical, an analog may be any structure that has the desired functionalities (including alterations or substitutions in the core moiety), even if comprised of different atoms or isomeric arrangements.

As used herein, the phrases “associated with” and “coupled to” include both direct association/binding of two moieties to one another as well as indirect association/binding of two moieties to one another. Non-limiting examples of associations couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

As used herein, “substantially pure” means an object species is the predominant species present (Le., on a molar basis it is more abundant than any other individual species in the composition), and a substantially purified fraction is a composition wherein the object species comprises at least about 50 percent (on a molar basis) of all macromolecular species present. Generally, a substantially pure composition will comprise more than about 80 percent of all macromolecular species present in the composition, such as (but not limited to) more than about 85%, 90%, 95%, and 99%. In a particular (but non-limiting) embodiment, the object species is purified to essential homogeneity (contaminant species cannot be detected in the composition by conventional detection methods), wherein the composition consists essentially of a single macromolecular species.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “pharmaceutically-acceptable excipient” refers to any carrier, vehicle, and/or diluent known in the art or otherwise contemplated herein that may improve solubility, deliverability, dispersion, stability, and/or conformational integrity of the compositions disclosed herein,

The term “patient” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof, or an amount of a treatment protocol (i.e., an alternating electric field), sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one tumor and/or cancer. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concept(s), certain non-limiting embodiments of the present disclosure are directed to compositions that comprise at least one Organoantimony(V) cyanoximate compound. In particular (but non-limiting) embodiments, the organoantimony(V) cyanoximate compounds of the present disclosure possess one or more non-antibiotic, antimicrobial activities.

Certain non-limiting embodiments of the present disclosure are directed to a composition that comprises at least one organoantimony(V) cyanoximate compound of the formula:

SbR₄₋ₙL_{y},

wherein R comprises at least one of an aliphatic or an aromatic group, and L is a cyanoximate group, and wherein (4−n)+y=5. That is, when n is 0, L is 1; when n is 1, L is 2; when n is 2, L is 3; and when n is 3, L is 4.

The R group of the organoantimony(V) cyanoximate compound may be any aliphatic or aromatic group known in the art or otherwise contemplated herein, so long as the organoantimony(V) cyanoximate compound can function as described herein.

For example (but not by way of limitation), the R group of the organoantimony(V) cyanoximate compound may comprise an aromatic group (such as, but not limited to, a phenyl group (C₆H₅), a benzyl group, a pyridine group, a pyrimidine group, or an aromatic group substituted with at least one of a halogen, alkyl, alkoxy, nitro-, and/or sulfo-group); in addition, n may be 0, and y may be 1.

In another non-limiting example, the R group of the organoantimony(V) cyanoximate compound may comprise an aliphatic group (such as but not limited to, a methyl group or other straight or branched chairs hydrocarbon group); in addition, n may be 1 or 2, and y may be 2 or 3.

Any cyanoxime molecules known in the art or otherwise contemplated herein may be utilized to produce the organoantimony(V) cyanoximate compounds of the present disclosure. In certain non-limiting embodiments, the cyanoxime molecule is selected from one of the following structures:

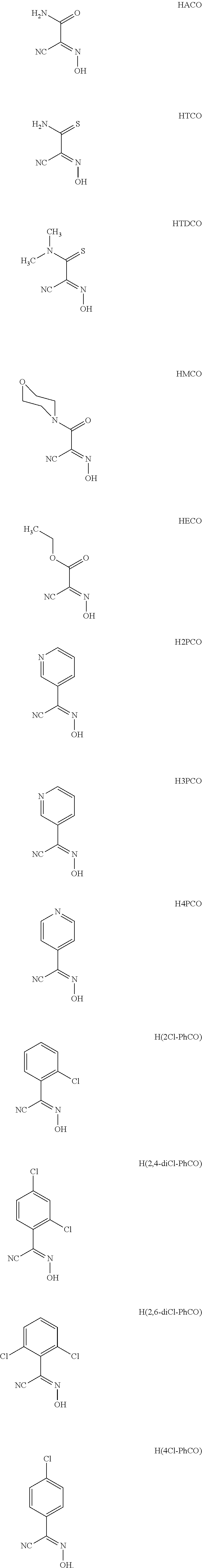

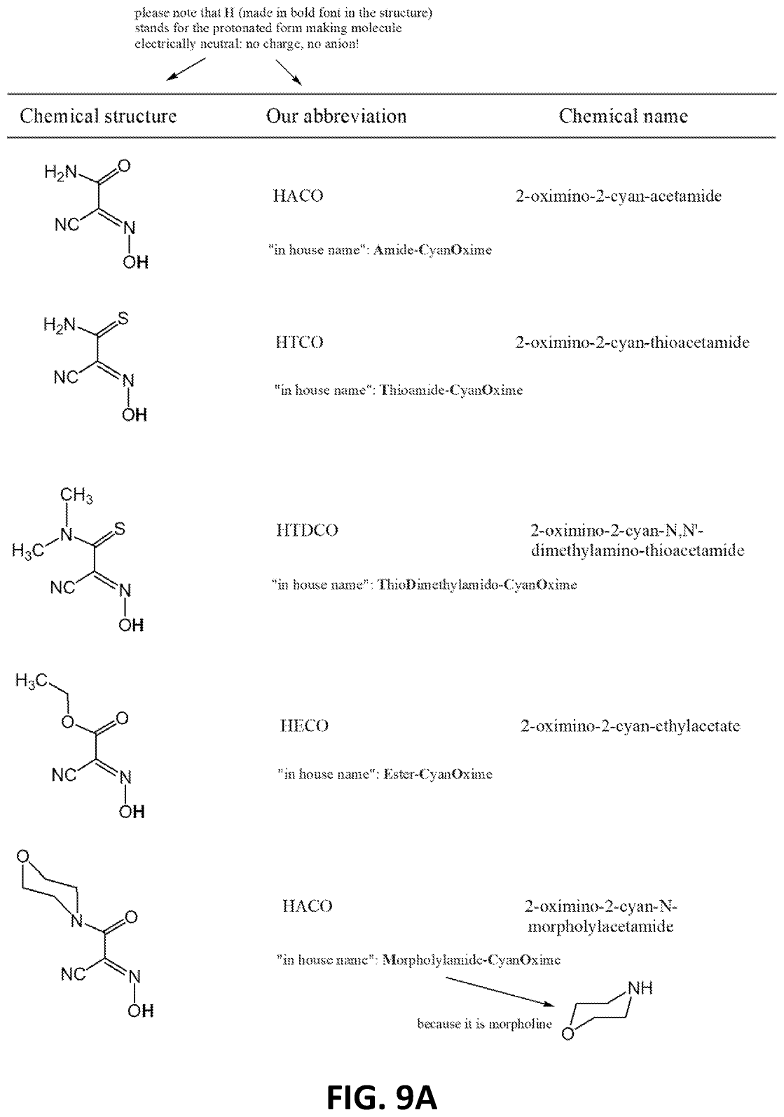

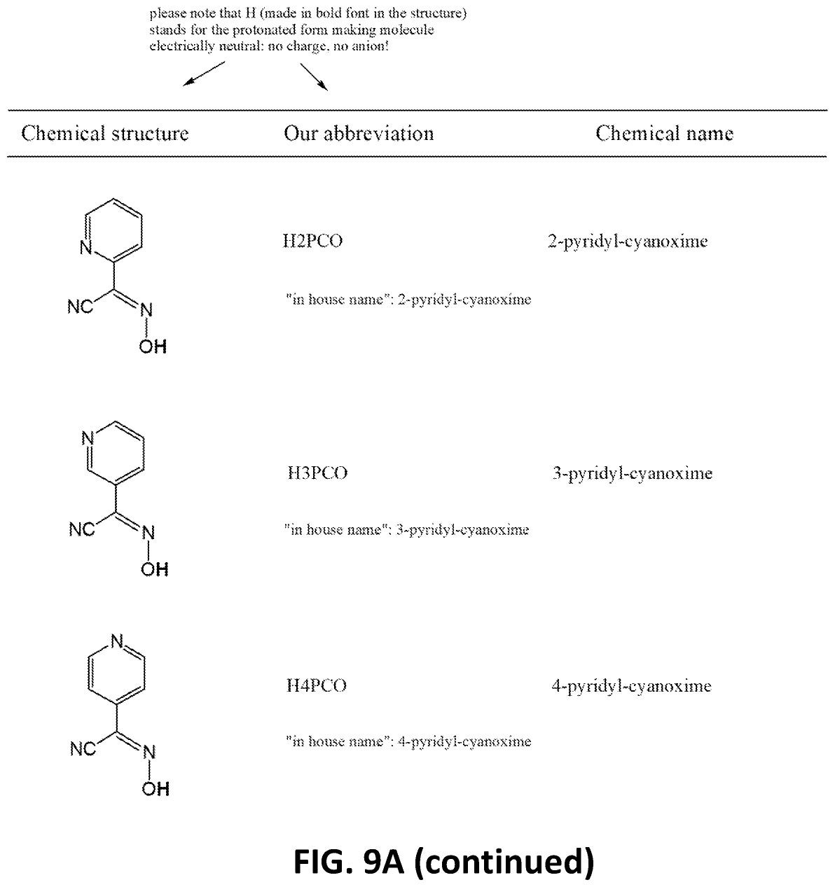

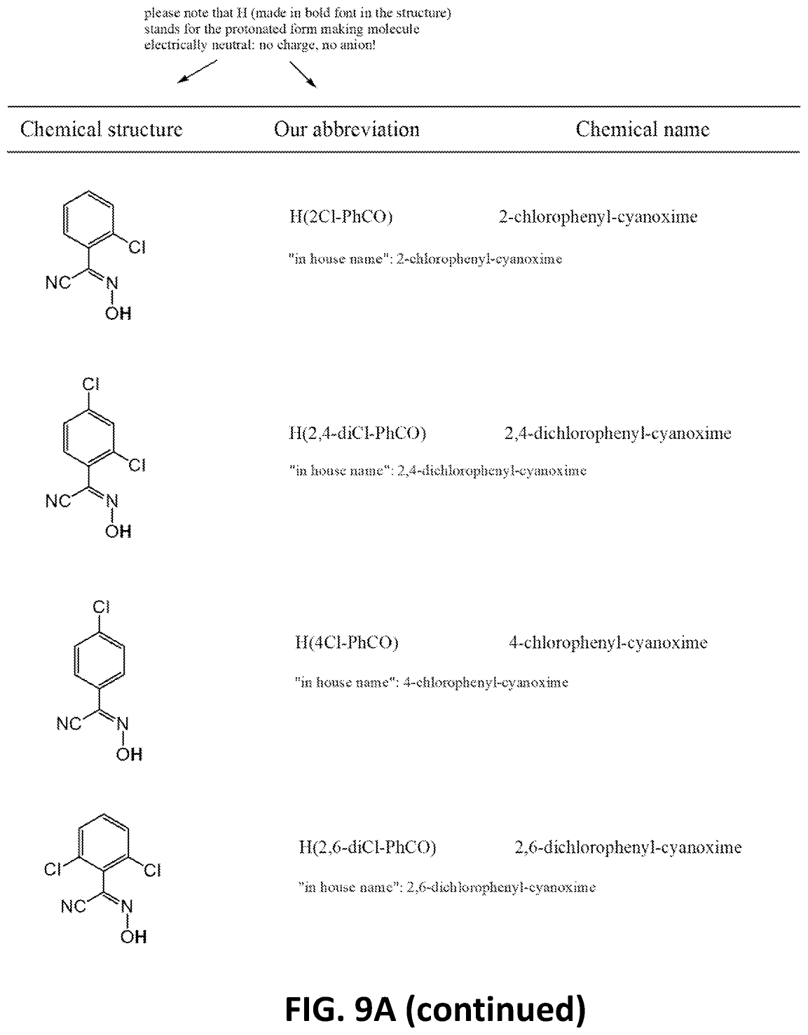

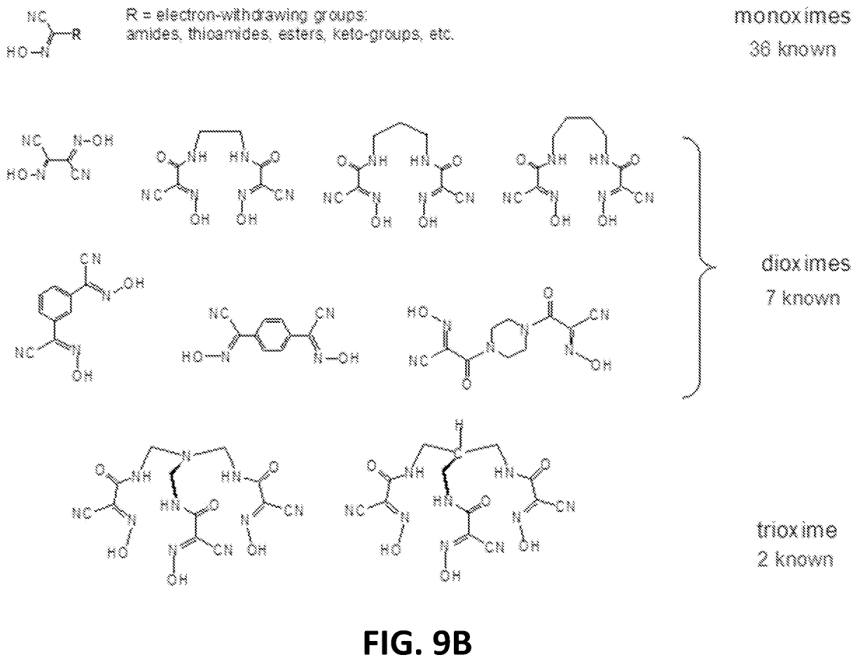

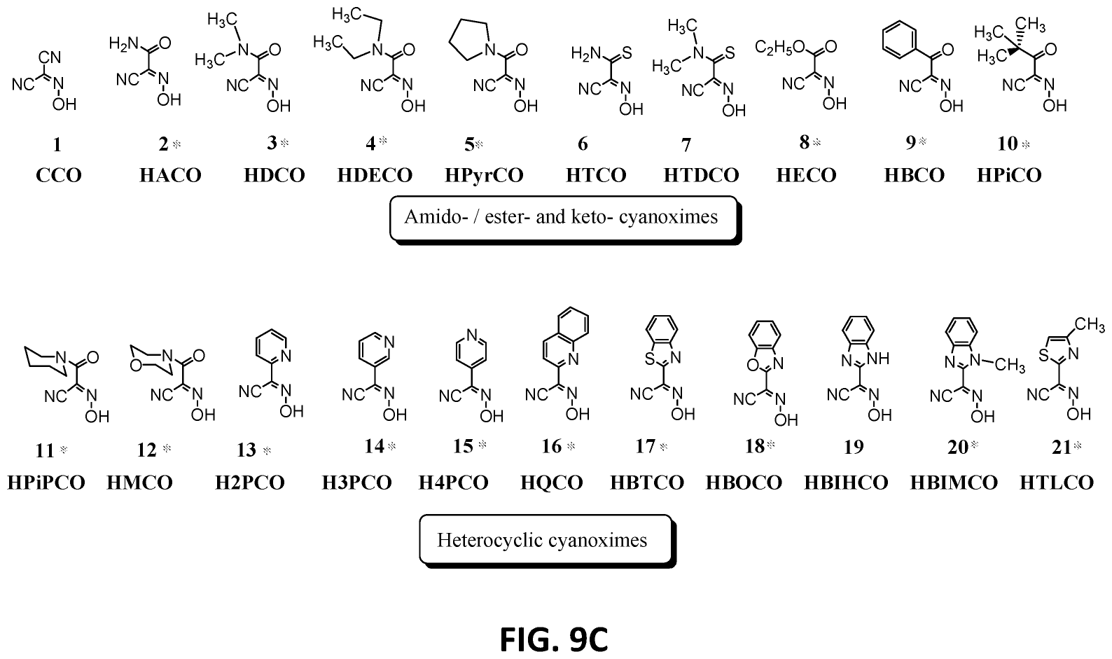

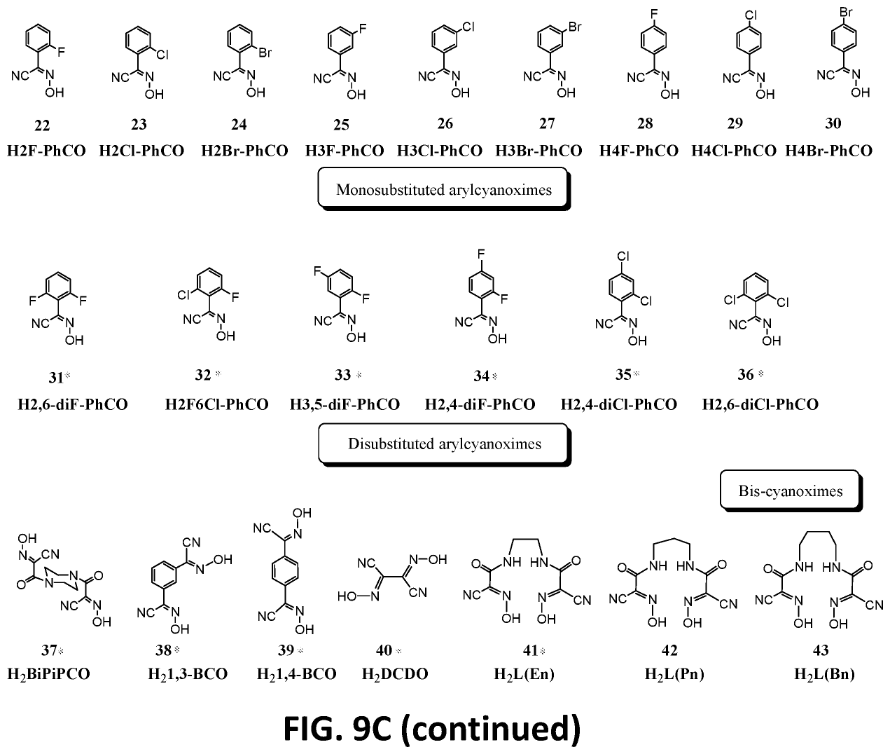

In addition, the cyanoxime molecule utilized to produce the organoantimony(V) cyanoximate compounds of the present disclosure may be selected from any of the cyanoximes illustrated in FIGS. 9A, 9B, and 9C.

In certain particular (but non-limiting) embodiments, the organoantimony(V) cyanoximate compound has one of the following structures:

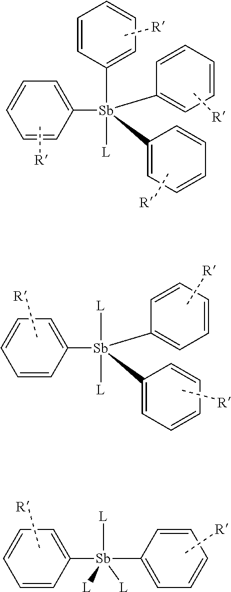

wherein each R′ comprises an aromatic group (such as, but not limited to, a phenyl group (C₆H₅), a benzyl group, a pyridine group, a pyrimidine group, or an aromatic group substituted with at least one of a halogen, alkyl, alkoxy, nitro-, and/or sulfo-group). Each L group may be selected from any of the cyanoximates disclosed or otherwise contemplated herein (such as, but not limited to, any of the cyanoximates of FIGS. 9A, 9B, and 9C), In certain particular (but non-limiting) embodiments, each L is 2PCO, 3PCO, 4PCO, ECO, ACO, MCO, TCO, or TDCO.

In particular (but non-limiting) embodiments, the organoantimony(V) cyanoximate compound has a generalized chemical structure represented by one of Formulas G1, G2, or G3 disclosed in the Examples section,

In certain particular (but non-limiting) embodiments, the organoantimony(V) cyanoximate compound has one of the following structures:

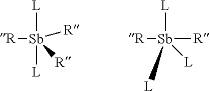

wherein each R″ comprises an alkyl chain (such as but not limited to, a methyl group or other straight or branched chain hydrocarbon group) and/or a cyclic alkyl group. Each L group may be selected from any of the cyanoximates disclosed or otherwise contemplated herein (such as, but not limited to, any of the cyanoximates of FIGS. 9A, 9B, and 9C). In certain particular (but non-limiting) embodiments, each L is ACO, NICO, ECU, TCO, TDCO, or a halogenated (F, Cl) arylcyanoxime.

In a particular (but non-limiting) embodiment, the organoantimony(V) cyanoximate compound has the structure:

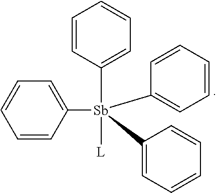

Each L group may be selected from any of the cyanoximates disclosed or otherwise contemplated herein (such as, but not limited to, any of the cyanoximates of FIGS. 9A, 9B, and 9C). In certain particular (but non-limiting) embodiments, each L is 2PCO, 3PCO, 4PCO, ECO, ACO, MCO, TCO, or TDCO.

In another particular (but non-limiting) embodiment, the organoantimony(V) cyanoximate compound has the structure:

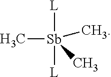

Each L group may be selected from any of the cyanoximates disclosed or otherwise contemplated herein (such as, but not limited to, any of the cyanoximates of FIGS. 9A, 9B, and 9C). In certain particular (but non-limiting) embodiments, each L is ACO, MCO, ECO, TCO, TDCO, or a halogenated arylcyanoxime.

In certain non-limiting embodiments, the organoantimony(V) cyanoximate compounds of the present disclosure possess antimicrobial activity. Non-limiting examples of antimicrobial activities possess by the organoantimony(V) cyanoximate compounds of the present disclosure include antibacterial activity and/or antifungal activity.

In a particular (but non-limiting) embodiment, the organoantimony(V) cyanoximate compounds of the present disclosure are effective at reducing, inhibiting, and/or substantially preventing growth of at least one bacterium and/or at least one fungus. Non-limiting examples of bacteria that the compounds of the present disclosure are effective against include Gram-negative bacteria such as (but not limited to) Escherichia species (i.e., E. coli, etc.), Pseudomonas species (i.e., P. aeruginosa, etc.), Salmonella species (i.e., S. typhimurium, etc.), Klebsiella species (i.e., K. pneumoniae, etc.), Acinetobacter species (i.e., A. baumannii, etc.), Campylobacter species, Cholera species, Haemophiius influenzae, Enterobacter species, Citrobacter species, Yersinia species, Shigella species, and the like; as well as Gram-positive bacteria such as (but not limited to) Staphylococcus species (i.e., S. aureus, S. epidermidis, etc.), Streptococcus species (i.e., S. pneumoniae, S. pyogenes, S. agalactiae, etc.), Enterococci, Corynebacterium diphtheria, Listeria monocytogenes, and the like. In a particular (but non-limiting) example, the bacteria may be a Methicillin-resistant strain. Non-limiting examples of fungi that the compounds of the present disclosure are effective against include Candida species (i.e., C. albicans, etc.)., Cryptococcus species (i.e., C. neoformans, etc.), Saccharomyces cerevisiae, Histoplasma species (i.e., H. capsulatum, etc.), Aspergillus species (i.e., A. fumigatus, etc.), and the like.

The compositions of the present disclosure may contain at least one organoantimony(V) cyanoximate compound as described or otherwise contemplated herein. However, it will be understood that the compositions may contain multiple organoantimony(V) cyanoximate compounds, such as two organoantimony(V) cyanoximate compounds, three organoantimony(V) cyanoximate compounds, four organoantimony(V) cyanoximate compounds, five organoantimony(V) cyanoximate compounds, six organoantimony(V) cyanoximate compounds, seven organoantimony(V) cyanoximate compounds, eight organoantimony(V) cyanoximate compounds, nine organoantimony(V) cyanoximate compounds, ten organoantimony(V) cyanoximate compounds, or more.

In addition, each organoantimony(V) cyanoximate compound may be present in the composition at any concentration that allows the organoantimony(V) cyanoximate compound(s) to function in accordance with the present disclosure (i.e., as an antimicrobial agent). Non-limiting examples of concentrations include a percent concentration of about 0.001%, about 0.005%, about 0.01%, about 0.05%, about 0.1%, about 0.5%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%. In addition, the scope of the presently disclosure also includes the presence of each organoantimony(V) cyanoximate compound in the composition at any percent concentration that falls within any range formed from the combination of two values listed above (for example, a range of from about 0.1% to about 99%, a range of from about 2% to about 80%, a range of from about 3% to about 60%, a range of from about 10% to about 95%, a range of from about 40% to about 75%, etc.).

While specific structures of organoantimony(V) cyanoximate compounds are disclosed and discussed in detail herein, it will be understood that specific modifications of the organic components of the compounds can be made to enhance their antimicrobial effect, For example (but not by way of limitation), specific modifications of the organic compounds can provide better water solubility, increase the amount of oxime groups attached to antimony atoms, etc. In addition, it is within the skill of a person of ordinary skill in the art to modify the organic components to arrive at these enhanced antimicrobial effects; therefore, said modifications fall within the scope of the present disclosure.

The compositions of the present disclosure have several applications, First, these compositions can be utilized in the development of new non-antibiotic antimicrobial drug compositions. Second, the compositions can be utilized as mucus-type antimicrobial hydrophobic additives, such as, but not limited to, additives for adhesives working in an aqueous environment, Third, these compositions can be incorporated into the production of antimicrobial wearable sweat resistant electronic devices (such as, but not limited to, for electrodes, contact pads, and the like). Fourth, these compositions can be utilized as an antimicrobial polymer glue (for example, but not by way of limitation, an antimicrobial polymer glue that sticks to wet surfaces for under water applications).

Certain non-limiting embodiments of the present disclosure are directed to a composition that comprises at least one of any of the compositions disclosed or otherwise contemplated herein disposed or otherwise incorporated within an adhesive.

Certain non-limiting embodiments of the present disclosure are directed to a pharmaceutical composition that comprises at least one of any of the compositions disclosed or otherwise contemplated herein and a pharmaceutically-acceptable excipient or carrier.

Any carriers or excipients known in the art may be utilized in accordance with the present disclosure. For example (but not by way of limitation), a physiological compatible carrier (e.g., saline) that is compatible with maintaining the structure/activity of the organoantimony(V) cyanoximate compounds when administered, and compatible with the desired mode of administration, may be utilized as the pharmaceutically acceptable carrier in accordance with the present disclosure. In addition, the active ingredients may be mixed with excipients which are pharmaceutically acceptable and compatible with the active ingredients. Suitable excipients include, for example but not by way of limitation, water, saline, dextrose, glycerol, ethanol, and the like, or any combination thereof.

The preparation of such compositions for use as therapeutic compositions is well known to those of skill in the art. Such compositions may be prepared either as liquid solutions or suspensions, or as solid forms, such as (but not limited to) tablets, pills, powders, and the like. Solid forms suitable for solution in, or suspension in, liquids prior to administration may also be prepared. The preparation may also be emulsified, In addition, the pharmaceutical compositions disclosed or otherwise contemplated herein may contain minor amounts of auxiliary substances, such as (but not limited to) wetting or emulsifying agents, pH buffering agents, and the like, as well as any combination thereof. If it is desired to administer an oral form of the pharmaceutical composition, one or more of various thickeners, flavorings, diluents, emulsifiers, dispersing aids, binders, or the like, as well as any combination thereof, may be added. The pharmaceutical compositions of the present disclosure may contain any such additional ingredients so as to provide the composition in a form suitable for administration.

The organoantimony(V) cyanoximate compound(s) may be present in the pharmaceutical composition at any percentage of concentration that allows the organoantimony(V) cyanoximate compound(s) to function as described or as otherwise contemplated herein, For example (but not by way of limitation), the organoantimony(V) cyanoximate compounds) may be present in a sufficient amount to provide an antimicrobial effect. In certain particular (but non-limiting) embodiments, each organoantimony(V) cyanoximate compound present in the pharmaceutical composition may be present at a percent concentration of about 0.001%, about 0.005%, about 0.01%, about 0.05%, about 0.1%, about 0.5%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%. In addition, the scope of the presently disclosure also includes the presence of each organoantimony(V) cyanoximate compound in the pharmaceutical composition at any percent concentration that falls within any range formed from the combination of two values listed above (for example, a range of from about 1% to about 99%, a range of from about 2% to about 80%, a range of from about 3% to about 60%, a range of from about :10% to about 95%, a range of from about 40% to about 75%, etc.).

Likewise, a pharmaceutically acceptable carrier and/or excipient may be present in the pharmaceutical composition at any percentage of concentration that allows the carrier/excipient to function as described or as otherwise contemplated herein. In certain particular (but non-limiting) embodiments, each pharmaceutically acceptable carrier and/or excipient is present in the pharmaceutical composition at a percent concentration of about 0.001%, about 0.005%, about 0.01%, about 0.05%, about 0.1%, about 0.5%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 99%. In addition, the scope of the presently disclosure also includes the presence of each pharmaceutically acceptable carrier and/or excipient in the pharmaceutical composition at any percent concentration that falls within any range formed from the combination of two values listed above (for example, a range of from about 1% to about 99%, a range of from about 2% to about 80%, a range of from about 3% to about 60%, a range of from about 10% to about 95%, a range of from about 40% to about 75%, etc.).

The pharmaceutical compositions of the present disclosure may be administered by any of the many suitable means described herein and/or which are well known to those of skill in the art, including but not limited to: by inhalation, intrapulmonary, intranasal, oral, injection (such as, but not limited to, intramuscular, intraperitoneal, intravitreal, or intravenous), or intradermal administration; and the like, In one instance, the administration will be carried out by using an implant (i.e., a device as described herein).

One or more than one route of administration can be employed either simultaneously or partially or wholly sequentially. Those of skill in the art are well acquainted with the planning, implementation, and assessment of antimicrobial dosing strategies, and therefore no further discussion thereof is required.

The pharmaceutical compositions may be administered in conjunction with other pharmaceutical compositions containing other antimicrobial compositions constructed in accordance with the present disclosure and/or other treatment modalities. In some embodiments, such additional treatment modalities may include (but are not limited to) various substances that boost the immune system, various antibiotic or antimicrobial agents, vitamins, anti-allergy agents, anti-inflammatory agents, etc. When multiple compositions/agents are to be administered together, the compositions/agents may be combined in a single pharmaceutical composition. Alternatively (and/or in addition thereto), the multiple compositions/agents may be administered separately but over a short time interval, e.g., at a single visit at a doctor's office or clinic, etc.

Certain non-limiting embodiments of the present disclosure are directed to a method of synthesizing at least one organoantimony(V) cyanoximate compound. In the method, an organoantimony(V) compound is combined with a cyanoxime to form a mixture, and the mixture is incubated under conditions whereby a metathesis reaction occurs and an organoantimony(V) cyanoximate compound is formed. Any of the cyanoxime molecules and any of the organoantimony(V) compounds disclosed or otherwise contemplated herein may be utilized in this synthesis method.

Each of the synthesis methods described or otherwise contemplated herein may produce the organoantimony(V) cyanoximate compounds with any level of yield, For example (but not by way of limitation), the non-antibiotic antimicrobial compounds can be synthesized with a yield of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, and at least about 99%. In addition, the scope of the presently disclosure also includes the production of the compounds at any percent yield that falls within any range formed from the combination of two values listed above (for example, a range of from about 10% to about 99%, a range of from about 30% to about 98%, a range of from about 50% to about 97%, a range of from about 60% to about 96%, a range of from about 70% to about 95%, etc.).

Certain non-limiting embodiments of the present disclosure are directed to antimicrobial compositions containing organoantimony(V) cyanoximate compounds produced by any of the methods described or otherwise contemplated herein.

Certain non-limiting embodiments of the present disclosure are directed to an assembly that includes a device having at least one surface and any of the compositions disclosed or otherwise contemplated herein incorporated within the device and/or applied to at least a portion of the at least one surface of the device.

The compositions of the present disclosure can be utilized with any types of devices for which the compositions can be coated thereon and/or incorporated therein and that could benefit from antimicrobial properties of the compositions, The antimicrobial composition(s) may be mixed with the materials from which the device is formed prior to extrusion/molding, so as to incorporate the antimicrobial composition within the device. Alternatively, the antimicrobial composition(s) may be deposited on a surface of the device after the device is formed.

Certain non-limiting examples of devices that can be utilized in accordance with the present disclosure include electrodes, contact pads, and the like. Another non-limiting example of a type of device that can be utilized in accordance with the present disclosure is a biomedical implant. The biomedical implant may be any device for implantation within a patient and for which antimicrobial properties on the surface thereof may be desired. Non-limiting examples of biomedical implants that can be utilized in accordance with the present disclosure are orthopedic devices, vascular prosthetic devices, endoprosthetic devices, arterial stents, neural probes, cardiac pacemakers, implanted cardiac defibrillators, intraocular lenses, intrauterine devices, breast implants, tympanostomy tubes, combinations thereof, and the like.

Certain non-limiting embodiments of the present disclosure are directed to a method of reducing, inhibiting, and/or substantially preventing microbial growth on a device. In the method any of the compositions disclosed or otherwise contemplated herein is applied to at least a portion of the surface of the device and/or incorporated within at least a portion of the device.

Certain non-limiting embodiments of the present disclosure are directed to a method that comprises administering an effective amount of any of the pharmaceutical compositions disclosed or otherwise contemplated herein to a patient in need thereof.

Any of the methods of the present disclosure may be utilized to reduce, inhibit, and/or substantially prevent growth of at least one bacterium and/or at least one fungus. The bacteria and/or fungi may be any of the bacteria/fungi described or otherwise contemplated herein, Particular non-limiting examples of bacterial infections/contaminations/colonizations that can be treated with the methods of the present disclosure include Escherichia coil, Pseudomonas aeruginosa, and Staphylococcus aureus . In a particular (but non-limiting) example, the bacteria may be a Methicillin-resistant strain. Particular non-limiting examples of fungal infections/contaminations/colonizations that can be treated with the methods of the present disclosure include Cryptococcus neoformans and Candida albicans .

### EXAMPLES

Examples are provided hereinbelow. However, the present disclosure is to be understood to not be limited in its application to the specific experimentation, results, and laboratory procedures disclosed herein. Rather, the Examples are simply provided as one of various embodiments and are meant to be exemplary, not exhaustive.

The Examples include the synthesis of three generations of novel organoantimony(V) cyanoximate compounds that include tetra-, tri-, and di-organic groups covalently bound to Sb(V) centers with one, two, or three L groups comprised of various cyanoxime moieties. The three generations of organoantimony(V) cyanoximate compounds are referred to herein as G1, G2, and G3 compounds, and the generalized chemical structures for each of these three generations of compounds are represented by the following Formulas.

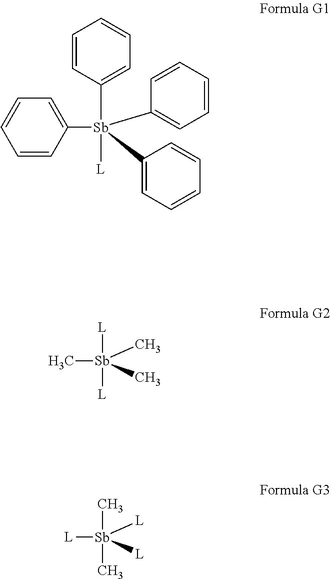

### Example 1

Chemical synthesis and characterization of organoantimony(V) cyanoximate compounds

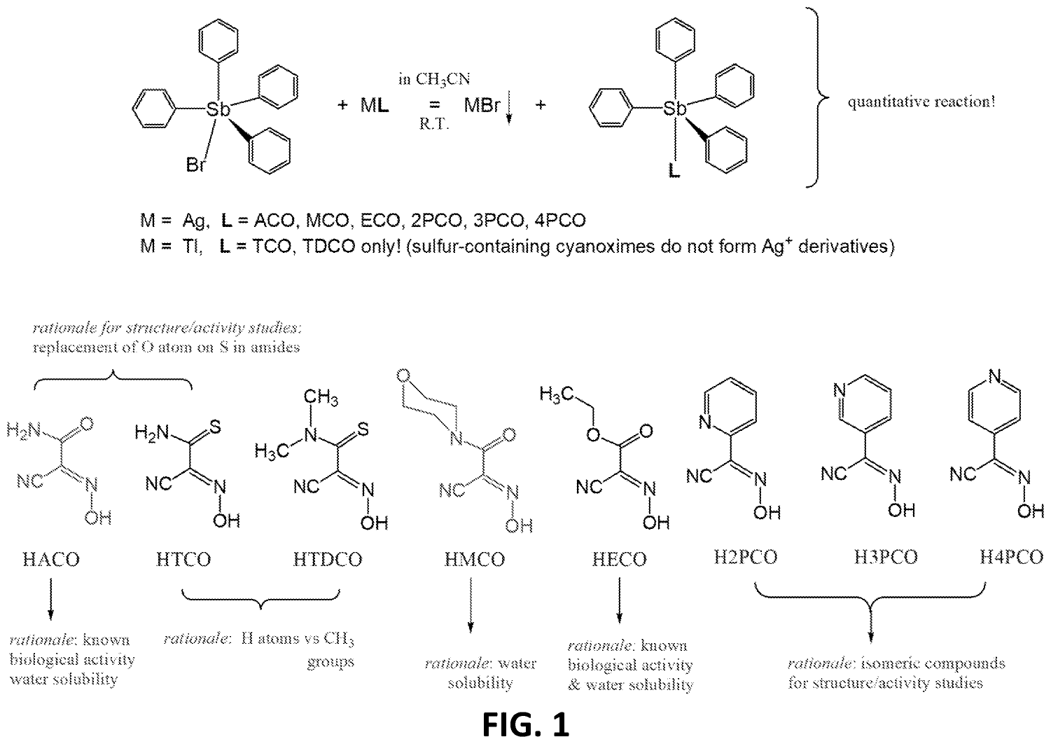

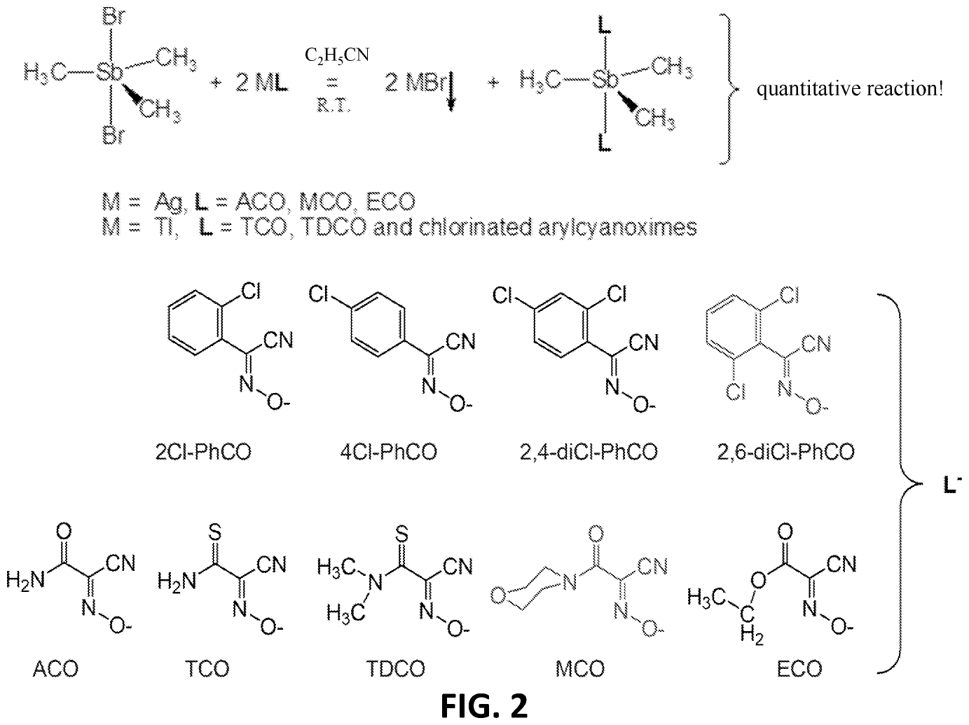

Utilizing a methodology for a high-yield preparation of a variety of cyanoximes from substituted acetonitriles R—CH₂—CN (U.S. Pat. No. 7,727,967; Cheadle et al., Dalton Trans (2013) 42(14):4931-46; Curtis et al., CrystEngComm (2012.) 15:152-159; Gerasimchuk et al., Inorganic Chemistry (1993) 38(6):964-970; and Mokhir et al., Inorganica Chimica Acta (1999) 284:85-98), the inventors had in their possession a library of 42 different cyanoximes that were obtained and studied in the Gerasimchuk research laboratory. The most recently synthesized group of compounds represents a new sub-class of oximes with general formula NC—C(═NOH)—R, where R is an electron-withdrawing group (Gerasimchuk et al., Current Inorganic Chemistry (2015) 5(1):38-63). These small molecules represent weak organic acids with a large variety of substituents R in their structure (Gerasimchuk et al., Dalton Transactions (2019) 48:7985-8013). Out of those 42 pure ligands, two groups of molecules were initially selected for cornplexation with organoantimony(V) moieties based on their known biological activity and solubility, as explained in FIGS. 1 and 2. The ligands shown in these figures possess interesting electronic and structural features such as hydrophilic/hydrophobic character, acidity, and bulkiness/steric demands that raise a question about the structure-activity relationship in biological studies, Two initial series of organoantimony(V) compounds, which were labeled as generation 1 (G1) and generation 2 (G2), were obtained with over >90% yield syntheses using a very efficient metathesis reaction (FIGS. 1-2).

There were 9 new organoantimony(V) cyanoximates obtained in each series. Comprehensive characterization of these compounds was carried out using elemental analyses, thin-layer chromatography (TLC), spectroscopic methods such as UV-visible, IR/Raman, multinuclear (¹H, ¹³C, ¹⁴N, ¹⁵N) NMR spectroscopy, thermal analysis, and X-ray crystallography available at the Chemistry Department at Missouri State University research facilities as well as off-campus services (elemental analysis, mass-specs). All the organoantimony(V) cyanoximate compounds thus far obtained represent mechanically and thermally stable on open air at ambient conditions crystalline solids soluble in common organic solvents.

Solid state structures and thermal properties of organoantimony(V) cyanoximate Compounds

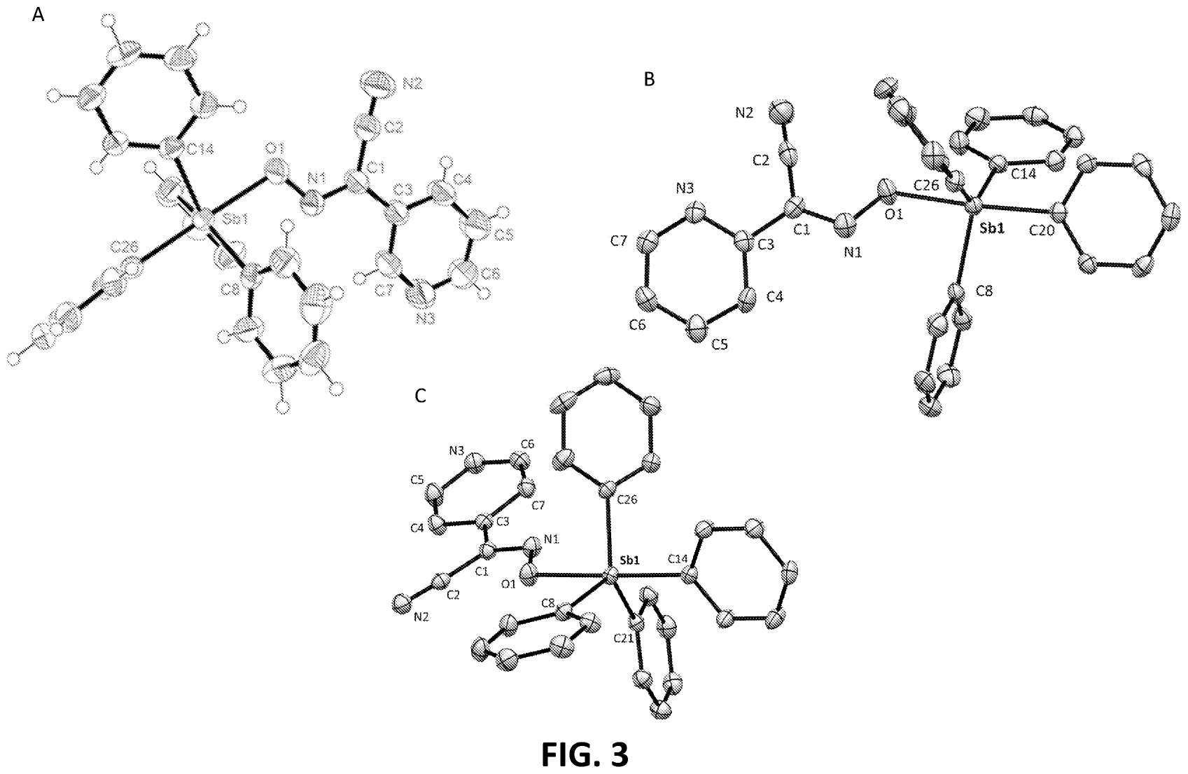

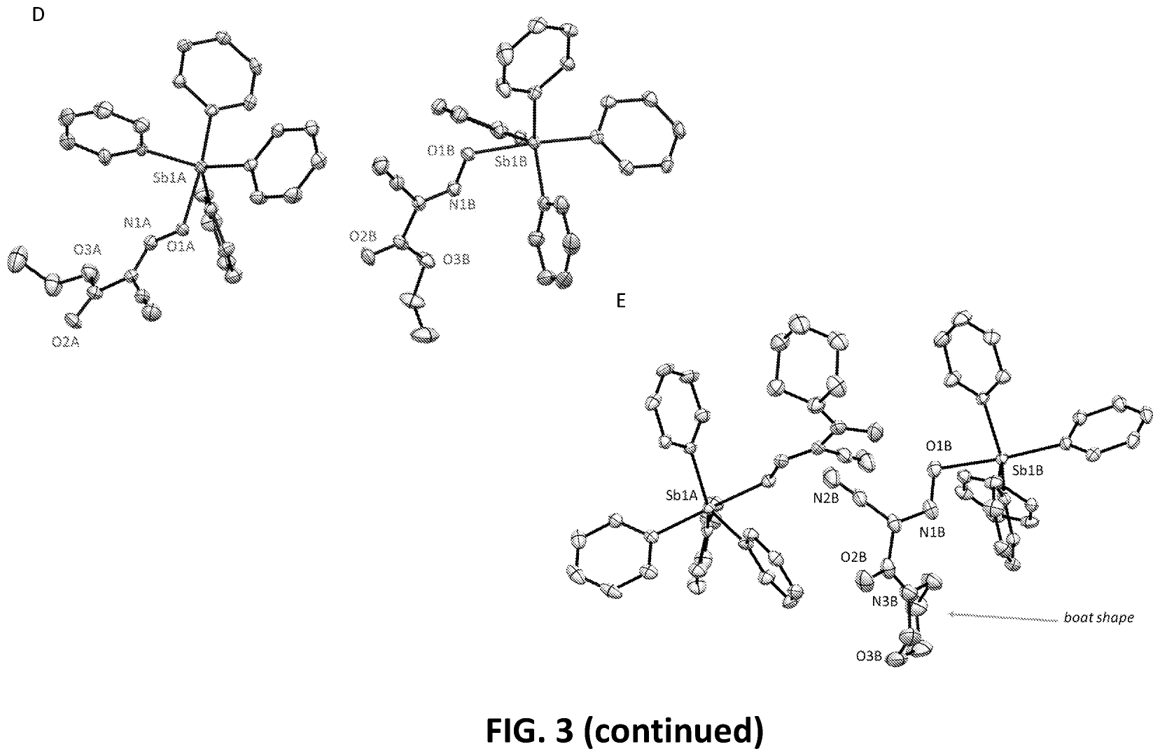

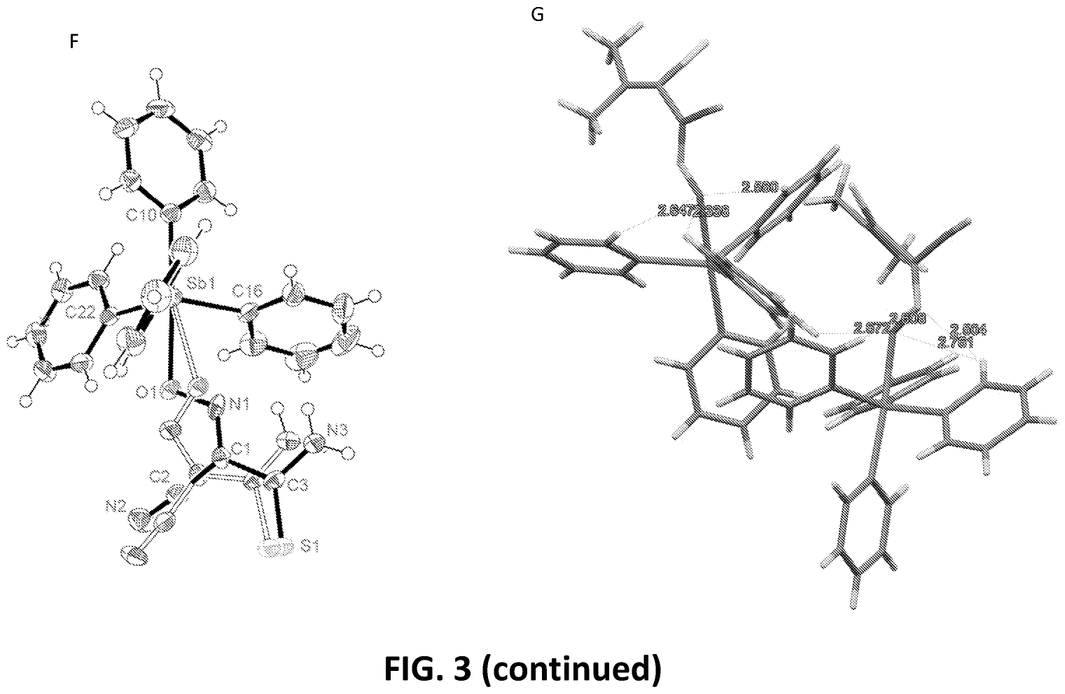

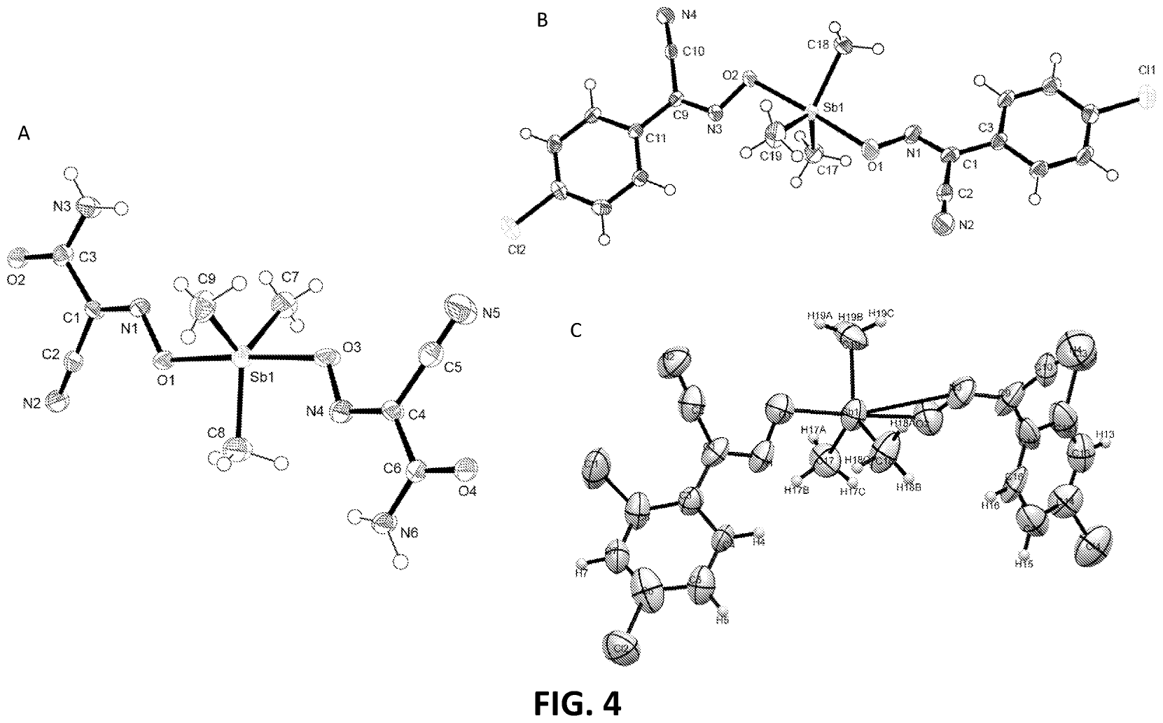

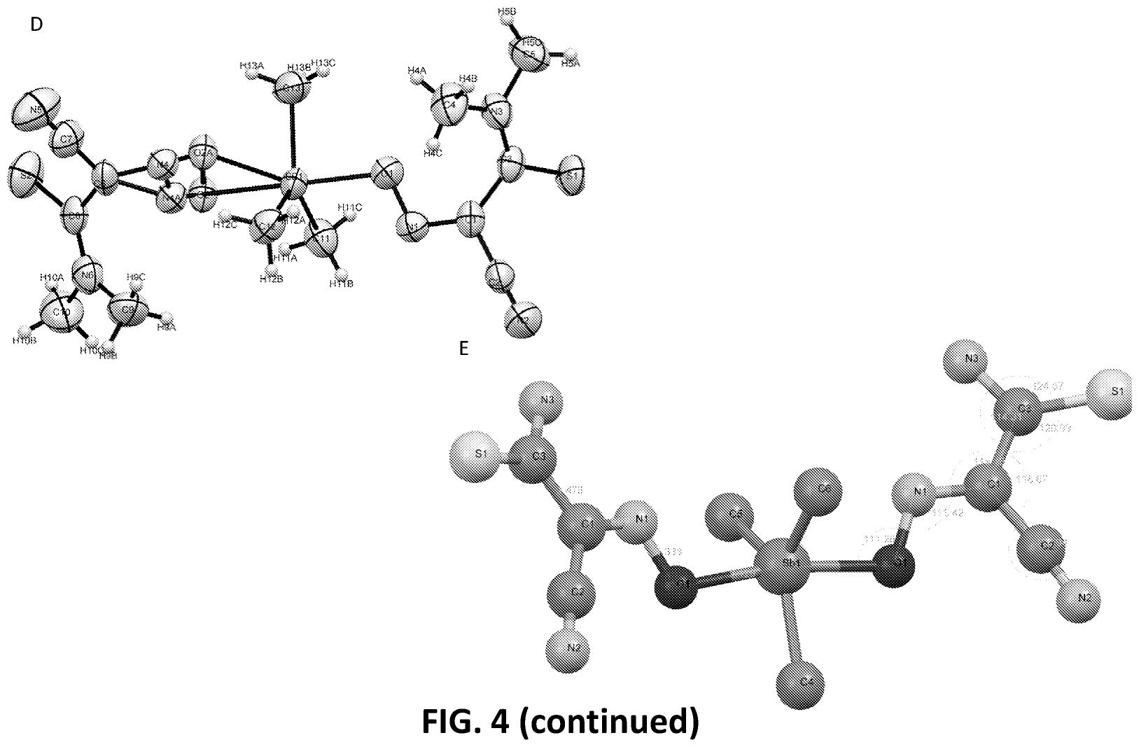

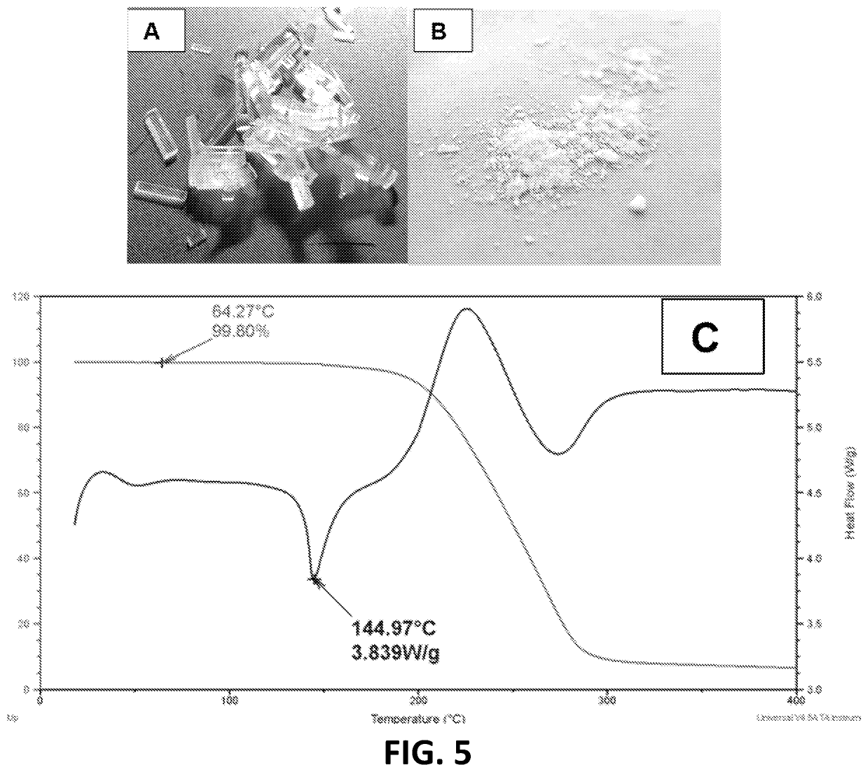

Crystal structures were determined for all 18 new antimonials in both G1 and G2 series (with exemplary structures shown in FIGS. 3-4). The crystal structures demonstrated that the central atom of Sb(V) adopts distorted trigonal pyramidal geometry with bound via O-atom in monodentate fashion cyanoxime anion (FIGS. 3-4). It was also demonstrated that all the newly synthesized organoantimonials were thermally stable up to ˜120° C. as evident from their thermograms, with two of them displayed in FIG. 5. All compounds melt without decomposition, which is a rare property for organornetallic compounds. This is important for potential use of these compounds for medical applications, as compounds used for therapeutic purposes should be mechanically and thermally stable, i.e., survive sterilization at 110° C. without decomposition.

Prior to the present disclosure, only a single report on the chemistry of organoantimony(V) cyanoximates was available (Domasevitch et al., Inorg Chem (2000) 39(6):1227-1237); there are, however, several publications and patents on the biological activity of cyanoximes, and particularly their antimicrobial activity, The data presented in the present disclosure demonstrates that organoantimony(V) cyanoximates constructed in accordance with the present disclosure possess a great potential as non-antibiotic antimicrobials.

### Assessment of Sb(V) Cyanoximates Antimicrobial Properties

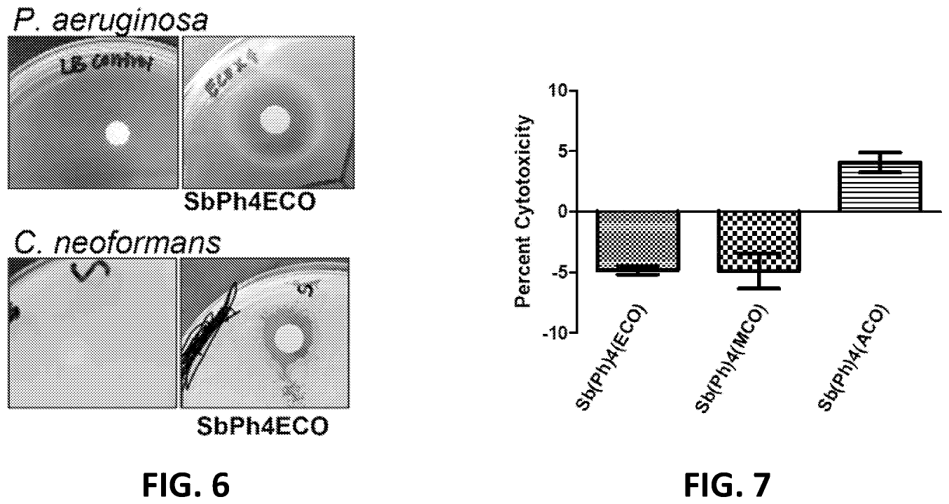

To determine the antimicrobial potential of the two generations (G1—based on SbPh₄-core, and G2—based on SbMe₃-core) of newly synthesized Sb(V) cyanoximates, several representative compounds were first selected and their antimicrobial properties tested by using disk diffusion assays, The minimal inhibitory concentrations (MIC) of selected compounds were also determined in liquid cultures. Three bacterial and two fungal pathogens were used as target microorganisms. Bacterial pathogens included Gram-negative Pseudomonas aeruginosa and Gram-positive Staphylococcus aureus, representing the ESKAPE group of human pathogens known for their high virulence and antibiotic resistance. The G1 SbPh₄-core compounds showed growth inhibition for P. aeruginosa (FIG. 6), S. aureus, and E. coli. The following MIC tests confirmed the plate results and demonstrated that five G1 compounds with the SbPh₄-core and at least three G2 compounds with the SbMe₃ -core completely inhibited growth of at least one of the pathogenic strains in the range from 50 to 100 μg/ml (Table 1). The corresponding organic backbone used as a control showed no inhibitory effect.

To determine the antifungal properties of the two groups of Sb(V) cyanoximaes, Cryptococcus neoformans and Candida albicans, both of which are deadly human pathogens, were tested. The disk diffusion assays showed that three of the first-generation compounds had measurable zones of inhibition when tested against C. neoformans and C. albicans (FIG. 6), The MIC assays showed that the same three compounds from the first generation inhibited the growth of both C. neoformans and C. albicans, Two of the compounds tested from the second generation inhibited the growth of C. neoformans, but none inhibited the growth of C. albicans (Table 1).

TABLE 1
The Minimum Inhibitory Concentrations (MIC) of the first two
generations of Sb(V) cyanoximates for Gram-negative bacteria
P. aeruginosa and E. coli, Gram-positive bacteria S. aureus,
and fungi C. neoformans and C. albicans

| G1 Compound, | P. | E. | S. | C. | C. |
|---|---|---|---|---|---|
| MIC, μg/ml | aeruginosa | coli | aureus | neoformans | albicans |
| SbPh4Br | >200 | >200 | 150 | 41.7 | 50 |
| SbPh4ACO | 100 | 100 | 100 | 18.8 | 15.6 |
| SbPh4ECO | 150 | >200 | 150 | 10.9 | 31.2 |
| SbPh4MCO | 200 | >200 | 100 | 19.8 | 32.1 |
| SbPh4TCO | >200 | >200 | 100 | 25 | 100 |
| SbPh4TDCO | >200 | >200 | 100 | 33.3 | 100 |
| SbMe₃Br₂ | 50 | 150 | 100 | >100 | >100 |
| SbMe₃(ECO)₂ | >200 | >200 | >200 | ND | ND |
| SbMe₃(MCO)₂ | 50 | >200 | >200 | ND | ND |
| SbMe₃(TCO)₂ | >200 | >200 | ND | ND | ND |
| SbMe₃(TDCO)₂ | >200 | >200 | ND | ND | ND |
| SbMe₃(2- | >200 | >200 | >200 | >100 | >100 |
| ClPhCO)₂ | | | | | |
| SbMe₃(2,4- | >200 | >200 | ND | >100 | >100 |
| ClPhCO)₂ | | | | | |
| SbMe₃(2,6- | >200 | 50 | ND | >100 | >100 |
| ClPhCO)₂ | | | | | |

Detected MICs in μg/ml are shown.
ND—not determined.

### Cytotoxicity Assessment.

In order to ultimately use the Sb(V) compounds as antimicrobial therapies, it must be confirmed that these compounds were not cytotoxic to mammalian cells. For this analysis, mouse cell line macrophages (J774) were incubated with three of the first-generation compounds that had activity against bacteria and fungi. These compounds were incubated at the highest MIC concentration for all organisms for 24 h with the J774 cells. Following incubation, the Vybrant cytotoxicity assay (ThermoFisher) was performed according to manufacturer's instructions. All compounds were found to be non-toxic (defined as less than 30% cytotoxicity) (FIG. 7).

### Example 2

Cyanoximes are known biologically active compounds that have recently been discovered to have pronounced enzyme inhibition activity (carbonic reductase), anticancer, and antimicrobial properties, They also regulate growth In plants and exhibit detoxifying properties on agricultural pesticides. The present disclosure is related to a combination of biologically active non-toxic metals and non-toxic organic ligands that leads to pronounced synergistic effects and opens new possibilities for practical applications of novel metallocomplexes. Antimony(V) cyanoximates are soluble in common organic solvents and can be obtained in high yields using a metathesis reaction from halogenated Sb(V) initial compounds and silver(I) cyanoximates (FIGS. 1-2), and were found to possess properties that make them useful for biomedical applications (Example 1).

Synthesis, identification, and further characterization of cyanoxime ligands.

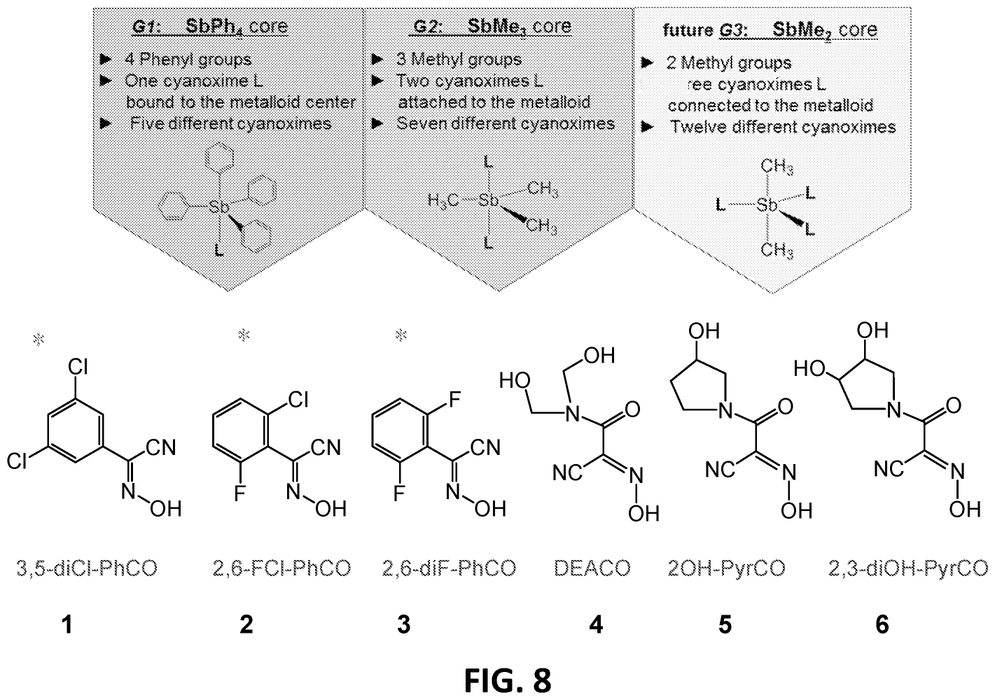

In this Example, new Sb(V) cyanoximates based on halo-aryl-cyanoximes and water-soluble amino alcohols are produced (FIG. 8) and are stable in a solid state and in solutions over a wide temperature range, which will enable their applications as antimicrobials.

Synthetic routes described in the literature (Gerasimchuk et al., Current Inorganic Chemistry (2015) 5(1):38-63; Gerasimchuk et al., Dalton Transactions (2019) 48:7985-8013) are used for the preparation of all cyanoximes shown in FIG. 8. The compounds are obtained using high-yield metathesis reactions between initial halogenated Sb(V) compounds and silver(I) cyanoxiomates in acetonitrile (FIG. 1) or propionitrile (FIG. 2) solutions. Target organoantimony(V) cyanoximates are soluble in organic solvents and can be easily separated from solid AgBr byproducts by centrifugation, followed by removal of solvent under vacuum and drying white crystalline compounds under vacuum.

The compounds are identified by using the TLC method, by elemental C, H, N content, and by measuring their melting points, Ail cyanoximes contain chromophore and exhibit fluorescence. In order to expedite the detailed characterization of these organic ligands and based on them antimony(V) compounds, cryostat enabling spectroscopic measurements (UV-visible, fluorescence) are used at variable temperatures. The range of thermal stability in solid state is determined using the TG/DSC method where temperatures and energies of thermal robustness are measured. These studies allow detection of stages of the new organoantimonials' decomposition and help to identify the final product of the process. All prepared thus far Sb(V) cyanoxirnates have UV-visible spectroscopic signatures that are used for investigation of the compounds' partitioning between organic and aqueous phases as well as for assessment of their stability at variable temperatures in solutions.

Characterize the antimicrobial efficacy and cytotoxicity of the third generation 12 Sb(V) cyanoximate compounds.

Evaluating the antimicrobial potential of novel compounds requires determining their minimum inhibitory concentrations (MIC). MICs define the spectrum of susceptible microorganisms and inform the relative efficacy of novel compounds in comparison to currently available means. In order to validate that new antimicrobial compounds can be considered for clinical applications, it is essential to determine whether the compounds would have a therapeutic window, i.e., their cytotoxic concentrations would be significantly higher than their MICs. This requires measuring their toxicity to mammalian cells. Data in Example 1 demonstrated that the first two generations of Sb(V) cyanoximates possess significant antimicrobial potential against both Gram-negative and Gram-positive bacterial pathogens as well as two species of pathogenic fungi. Further, selected compounds showed no cytotoxicity at the MIC levels. The third generation of Sb(V) cyanoximates further expands the library of compounds and allows for the identification of highly antimicrobial compounds with low cytotoxicity.

Based on the preliminary data shown in Example 1 that demonstrated that at least seven Sb(V) cyanoximates from the first two generations inhibit microbial growth in liquid and solid media, the new generation of Sb(V) cyanoximates that is based on a core with two methyl groups and three cyanoximes has a stronger potential in inhibiting growth of all or selected groups of bacterial and fungal pathogens. The Sb(V) cyanoximates increase the efficiency of existing antimicrobials but are not toxic to mammalian cells.

When testing new potential antimicrobials, it is important to select microorganisms that are clinically relevant. P. aeruginosa causes severe acute and chronic infections that are extremely difficult to treat and have high morbidity and mortality rates. In recent years, S. aureus has become the leading cause of infections related to indwelling medical devices such as vascular catheters, prosthetic joints, and artificial heart valves. Enterococcus faecium is a common resident of human gastrointestinal tracts capable of causing a variety of severe infections, most often among antibiotic-treated hospitalized patients with perturbed intestinal microbiota. These three bacteria belong to the ESKAPE group of human pathogens known for their high virulence and antibiotic resistance. Although most E. coli are harmless or constitute an important part of human normal microflora, some strains are pathogenic and may cause disease in humans and animals, These bacteria represent Gram-negative (P. aeruginosa and E. coli) and Gram-positive (S. aureus and E. faecium) groups that differ drastically in the structure of their cell walls and therefore in the ability to withstand antimicrobials and the mechanisms of resistance. Furthermore, these bacteria have been previously characterized as resistant to most available antibiotics and therefore require the development of new and efficient antimicrobials. They also form biofilms, communities of surface-associated microorganisms that are often encapsulated in extracellular polysaccharide matrix material, Biofilms show further increased resistance to antibiotics and antifungals and host defenses and therefore are notoriously difficult to eradicate and are a source of many recalcitrant infections. Therefore, the inhibitory effect of new Sb(V) cyanoximates of FIG. 8 are tested on growth of free-swimming and biofilm cultures of P. aeruginosa PAO1, S. aureus NRS70, E. faecium CC17, and E. coli S17 strains. For fungal organisms, the clinically relevant pathogens Cryptocaccus neoformans and Candida albicans have been chosen for testing. C. neoformans causes fatal meningitis in immune compromised patients and is the second-leading cause of death in AIDS patients, resulting in 180,000 deaths annually in AIDS patients alone. Therapies for cryptococcal meningitis usually involve Amphotericin B (AmpB), which is only effective in about 40% of meningitis patients and is known to cause nephrotoxicity. In addition, C. neoformans is becoming resistant to this and other available antifungal drugs. C. albicans causes mucosal infections but is also a prevalent nosocomial-acquired bloodstream pathogen. This invasive form of candidiasis is extremely difficult to treat, and the organism is becoming increasingly resistant to current antifungal drugs. C. albicans is also becoming very resistant to the azole drugs, and some Candida strains have also developed resistance against the newest class of antifungal drugs, the echinocandins. In addition, both fungal organisms can form biofilms, enhancing their pathogenicity, Therefore, the inhibitory effect of the new Sb(V) cyanoximates described in this Example is tested against these two fungal pathogens.

Minimal inhibitory concentrations (MIC) of Sb(V) cyanoximates for bacterial and fungal organisms.

MIC of the 3rd generation compounds, as well as complete MIC measurements for the 2nd generation Sb(V) compounds, are determined. A dilution assay in a 96-well-plate format, the gold standard for measuring MICS recommended by the Clinical Laboratory Standards Institute (CLSI), is utilized. Prior to plate inoculation, bacterial cultures of P. aeruginosa PAO1, S. aureus NRS70, E. foecium CC17, and E. coli S17 are grown in Mueller Hinton Broth (MHB) to middle log phase as determined by the individual growth curves and diluted to obtain absorbance at 600 nm of 0.1. These normalized cultures are diluted 1:100 using MHB and inoculated into the fresh medium with serially diluted Sb(V) compounds in 96-well plates. The plates are incubated for 24 h at 37° C. For fungal pathogens, cultures of Cryptococcus neoformans strain H99 and Candida albicans strain SC5314 are grown to stationary phase in YPD medium for 18 h at 30° C. Following incubation, fungal cells are centrifuged, washed three times with PBS, and then counted with trypan blue exclusion. Each compound is screened using a microdilution assay with 0.5×10³ fungal cells in RPMI-MOPS medium at 35° C. for 48 h. Following incubations for bacterial or fungal pathogens, the OD600 is measured using a Biotek Synergy HT microtiter plate reader (Tecan instruments Inc.). The MIC is defined as the lowest concentration that inhibits growth. An aliquot from the clear wells from the MIC assay is plated on agar to determine minimum bactericidal concentration (MBC) or minimum fungicidal concentration (MFC). Each experiment is based on at least three biological replicates and repeated for consistency. The MIC and MBC for each compound is compared to those obtained for clinically applied antibiotics/antifungals. For bacterial pathogens, the corresponding antibiotics, such as tobramycin (P. aeruginosa), trimethoprim (S. aureus), gentamicin (E. coli) and ampicillin (E. foecium), are used. For fungal pathogens, the data is compared to the antifungal drugs Amphotericin B and Caspofungin for C. neoformans and C. albicans, respectively, by using the CLSI method.

To measure MIC in biofilms of the compounds that showed activity in liquid cultures, the bacterial and fungal cultures are inoculated and grown as described above. Following incubation, the planktonic fractions are removed, and the remaining surface-associated cells (biofilms) are washed three times with saline (0.85% NaCl) to remove loosely attached cells and stained with crystal violet (0.1%, w/v) for 10 min. The dye is solubilized in 30% (v/v) acetic acid and 70% (v/v) ethanol, and the absorbance of the extracted crystal violet stain is measured at 595 nm. Non-inoculated wells are measured as blanks, and the corresponding measurements are subtracted from the rest of the data. The minimal concentrations of the compounds at which no biofilm growth is detected are reported as MIC. The data represents the mean values of at least three technical replicates and three biological replicates. Each experiment is repeated for consistency.

Antimicrobial synergy with the clinically used antibiotics.

it is determined whether Sb(V) compounds increase sensitivity to antibiotics antifungals by determining the MIC of the most efficient compounds in combination with the antibiotics/antifungals commonly used to treat the corresponding infections. For bacterial pathogens, P. aeruginosa, S. aureus, E. coli, and E. faecium, tobramycin, trimethoprim, gentamicin, and ampicillin, respectively, are used. The antifungal drugs most commonly used to treat C. neoformans and C. albicans are Amphotericin B and Caspofungin, respectively. For this, a checkerboard analysis is used, which allows for detection of synergy or antagonism between the compounds and antibiotics/antifungals. To quantify the interactions, the fractional inhibitory concentration (FIC) index value is calculated, which takes into account the MICs of the individual treatments (MICA and MICB) and their MICs when applied in combination (A and B). FIC=A/MIC_{A} +B/MIC_{B}. The experiments are performed in both planktonic and biofilm cultures. The strains are inoculated, and planktonic and biofilm growth are assayed as described above. The FIC<0.5 suggests synergy, and FIC>4 suggests antagonism.

Cytotoxicity. Prior to using these compounds in an infection model, the compounds are verified to be non-toxic to mammalian cells, For this, each compound is incubated with J774A.1 murine macrophages (5×10⁵/ml) in a 100 μl volume in a 96-well plate. These cells are incubated with each antimicrobial compound at the highest MIC value (highest for both bacteria and fungi) for 24 h followed by cytotoxicity analyses. Untreated cells serve as a negative control, and lysed cells serve as a positive control. The Vybrant cytotoxicity assay (ThermoFisher) is used according to manufacturer's instructions to calculate cytotoxicity, as described in Example 1. Compounds are classified as non-toxic if they have less than 30% cytotoxicity in the assay. Cytotoxicity analyses are conducted in triplicate wells, and each compound is tested in three independent experiments for rigor and reproducibility.

In vivo analysis using Galleria melonella animal model. To assess the efficacy of the most efficient compounds for treating infections in vivo, the Galleria mellonella infection model is used, which is routinely used for studying P. aeruginosa virulence, This model has been established for studying bacterial pathogenesis and fungal pathogens. Active larvae of 2-3 cm length are selected and stored in the dark at 4° C. without feeding for no more than 48 h before injection. P. aeruginosa PAO1, S. aureus NRS70, E. faecium CC17, and E. coli S17 are grown in MHB to middle log. Cultures are serially diluted to achieve two-five colony forming units (CFU) per injection. To verify the injected infection dose and effect of the treatment, the cultures are plated on MHB agar plates for CFU count. For fungal organisms, C. albicans or C. neoformans are grown for 16 h at 30° C. in YPD broth. Organisms are washed three times with sterile phosphate-buffered saline (PBS), and inocula of 1×10⁴ to 1×10⁶ organisms are prepared. After the gentle rinse with 70% ethanol followed by 1 mg/mL rifampicin, twenty larvae are injected with 5 μL of normalized treated cell suspensions through the last left pro-leg. After 1 h of incubation at 37° C., ten larvae are injected with 5 μL single dose of Sb(V) cyanoximate component of choice diluted in PBS at the MIC level directly into the central cavity (hemocoel), which closely mimics the conventional administration route used in mammalian models. The other ten infected larvae are injected with PBS alone to serve as untreated controls. In addition, five larvae are not infected and only injected with 5 μL of PBS, and the other five are injected with 5 μL of each Sb(V) cyanoximate compound in question diluted in PBS to be used to control for injection injuries and toxicity of Sb(V) cyanoximate compounds. The larvae are incubated and tested for death every 12 h for 72 h, which was selected to exclude the effect of cocooning that begins soon after. Times of death (TOD) are recorded for each larva, when no movement is observed in response to turning over with a sterile toothpick. Each experiment is repeated at least three times, and the survival rates are averaged and statistically analyzed for comparison by using GraphPad Prism 5 software. The Galleria model is also used to validate the antimicrobial synergy of the compounds that show positive results in vitro.

This Example allows for the identification of several Sb(V) cyanoximate compounds with high antimicrobial effect against at least selected microorganisms, wherein these compounds are not cytotoxic at the MIC levels and would enable recovering from infections in vivo. The identified compounds also have a synergistic impact when applied in combination with clinically applied antibiotics.

### Example 3

### Chemical Synthesis Design

FIG. 9A provides the chemical name and structure for certain cyanoximate compounds utilized in accordance with the present disclosure, FIG. 9B provides generalized chemical formulations for monoximes, dioximes, and trioximes of cyanoximes synthesized in accordance with the present disclosure, and FIG. 9B illustrates the chemical structures of various members of different classes of cyanoximes synthesized in accordance with the present disclosure. The H shown in bold in each structure stands for the protonated form, making the molecule electrically neutral, and the molecule is abbreviated as HL. During chemical synthesis of the organoantimony(V) cyanoximate compounds of the present disclosure, the proton is removed and replaced with a suitable metal center. Therefore, neutral HL molecule is derivatized into L—, and with an added monovalent metal cation such as (but not limited to) silver, sodium, thallium, the cyanoxime becomes AgL, NaL, or TIL These metal derivatives react quickly and almost quantitatively with an organoantimony moiety (such as, but not limited to, Ph₄SbBr or Me₃SbBr₂) to give Ph₄SbL and Me₃SbL₂ (FIG. 1).

This is a metathesis reaction (or double displacement) where all four components in reactants exchanged places, and the driving force for these clean reactions is the formation of insoluble silver bromide, AgBr. Centrifugation affords clear transparent solutions of tetraphenylantimony cyanoximates, from which the target compounds readily crystallize.

The formulas for certain organoantimony(V) cyanoximate compounds produced by the reaction shown in FIG. 1 are Ph₄Sb(ACO), Ph₄Sb(ECO), Ph₄Sb(MCO), etc. The hydrogen in the OH group of the cyanoxime will be removed to provide an O-group, because the cyanoximate is an anion and bound to the Sb-center that is positively charged.

Because of the fact that for sulfur-containing cyanoximes HTCO and HTDCO there are no silver salts [fast decomposition], a thallium(I) cation was used to prepare the anionic form of these two cyanoximes for the metathesis reaction of FIG. 1. Again, insoluble thallium halide is easily removable from the reaction mixture, leaving transparent yellow solutions of these two cyanoximates.

FIG. 1 illustrates the production of eight compounds of the formula Ph₄SbL that were synthesized and characterized using a full set of available spectroscopic and physical methods. These compounds included four very hydrophobic groups attached to the Sb(V) center and only one cyanoxime group per compound.

Another organoantimony derivative utilized was the Me₃Sb-fragment. In this formula, there are two more places where cyanoximes can be added. Therefore, these compounds have three organic groups (i.e., methyl groups) that are less hydrophobic and less bulky than the groups described in FIG. 1 in combination with two cyanoximes that complete pentavalent surrounding of Sb(V) centers.

FIG. 2 illustrates nine compounds based on this formula that were made and characterized. Crystal structures of all compounds were determined.

Halogenated cyanoximes have been shown to inhibit carbonyl reductase enzyme; this enzyme is the key protein that makes anticancer treatment inefficient, so blocking of that enzyme gives better chances in chemotherapy. Similar experiments have also been conducted with halogenated molecules that included fluorine derivatives.

### Example 4

The requirement of new antimicrobial treatments has become an urgent field in the last two decades. Multi-drug resistant (MDR) pathogenic bacteria and fungi are now resistant to most available antibiotics, including new generation, ceftazidime and daptomycin. The present Example allowed identification of several cyanoximes as biologically active compounds, as further investigated herein (FIG. 1).

To antagonize diverse bacteria and fungi, eight novel organoantimony(V) cyanoximates were: 1) synthesized using the metathesis reaction in dry acetonitrile between tetraphenylantimony(V) bromide or triemethylantimony(V) dibromide and Ag(I) or Tl(1) salts of several cyanoximes, 2) in vitro studied using several antibiotic resistant pathogenic microbes.

Synthesis: Desired organoantimony(V) cyanoximates obtained in the metathesis reaction were left in solution while AgBr or TlBr were separated off using centrifugation (FIG. 2). Removal of solvent in desiccator led to crystalline molecular compounds of SbPh₄L or SbMe₃L₂ composition (L=biologically active cyanoximes selected for investigations). Identity and structures of compounds were confirmed by elemental analysis on C, H, N, and S content, thermal analysis, IR-, ¹³C{¹H} NMR, some with UV-visible spectroscopy, and single crystal X-ray analysis. It was found that in all organoantimony(V) cyanoximates, the cyanoxime moiety is bound to the central atom via oxygen atom, and there is a formation of a lightly distorted trigonal bipyra mid polyhedron of the Sb(V) atom,

Two different types of antimicrobial studies were performed: 1) paper disk studies during growth on solid media; and 2) MIC determination during growth in liquid media. It was found that that Sb(Ph)₄ (ACO) and Sb(Ph)₄(ECO) had significant antimicrobial effect against all three strains selected for investigation: two Gram-negative strains—Escherichia coli strain S17 and Pseudomonas aeruginosa strain PAO1; and Gram-positive Methicillin-resistant Staphylococcus aureus strain NRS70. Compounds Sb(Ph)₄ (TCO) and Sb(Ph)₄ (TDCO) had significant effects on the Gram-positive Methicillin-resistant Staphylococcus aureus strain NRS70, but essentially no antimicrobial activity for the Gram-negative strains used. Antifungal disk assays concluded that Sb(Ph)₄ (ECO) was effective against Cryptococcus neoformans and Candida albicans. Sb(Ph)₄ (TCO) followed in antifungal activity against both strains. Sb(Ph)₄ (ACO) and Sb(Ph)₄ (TDCO) were only effective at inhibition of Cryptococcus neoformans. For MIC assays, Sb(Ph)₄ (MCO), the Sb(V) cyanoximate, was tested, along with the free cyanoximes H(MCO), H(ECO), and Na[H(ACO)₂]. As expected, free cyanoximes have no antimicrobial effect.

### Example 5

In this Example, antimicrobial activity of a series of new organoantimony(V) compounds constructed in accordance with the present disclosure was analyzed. This Example includes antimicrobial testing against notorious human pathogens, as well as antifungal testing. In both testing examples, solid powders of the compounds as well as solutions thereof were investigated.

First, solid state powder compounds were deposited on paper disks and tested against two fungi: Cryptococcus neoformans and Candia albicans. Each experiment was conducted in triplicate with each compound at each concentration of deposited solid compound. The control was a blank paper (100% cotton disk).

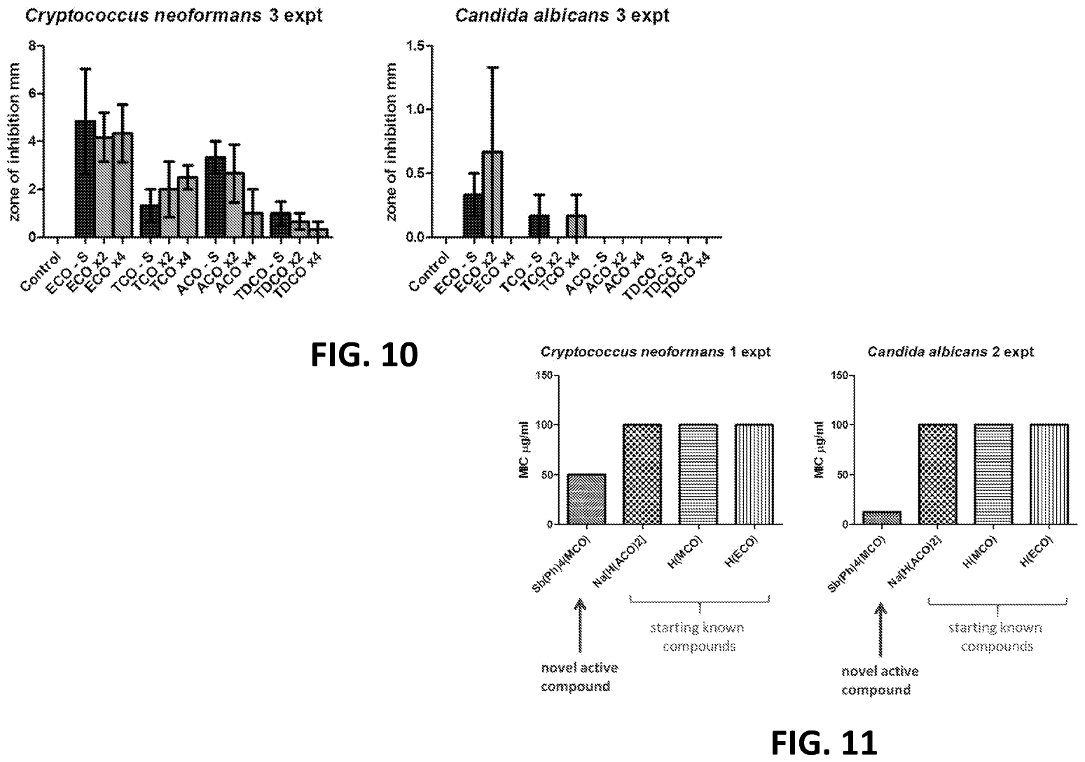

As shown in FIG. 10, all organoantimony(V) compounds tested exhibited antifungal activity against C. neoformans, while several of the organoantimony(V) compounds tested exhibited antifungal activity against C. albicans.

Second, the compounds were dissolved in DMSO to 5 μg/ml, and their minimal inhibitory concentration (MIC) was determined against C. neoformans and C. albicans. Starting concentration used was 100 μg/ml, and then diluted 1:2. A compound is considered to have antifungal activity if the MIC is below 100 μg/ml (for reference, the MIC concentration of the antifungal drug Amphotericin B is 0.5 μg/ml).

As can be seen in FIG. 11, the novel active compound Sb(Ph)₄(MCO) exhibited antifungal activity against both C. neoformans as well as C. albicans, whereas none of the three known starting compounds possessed any antifungal activity.

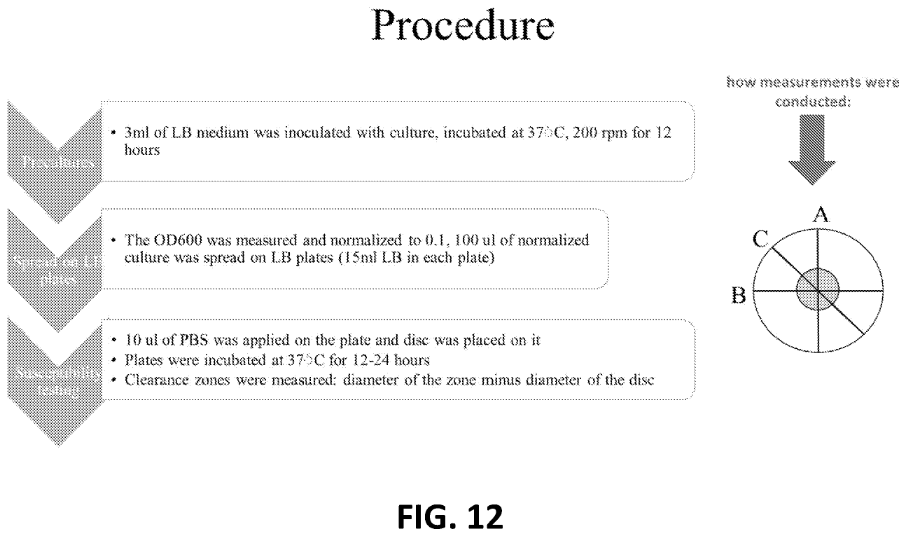

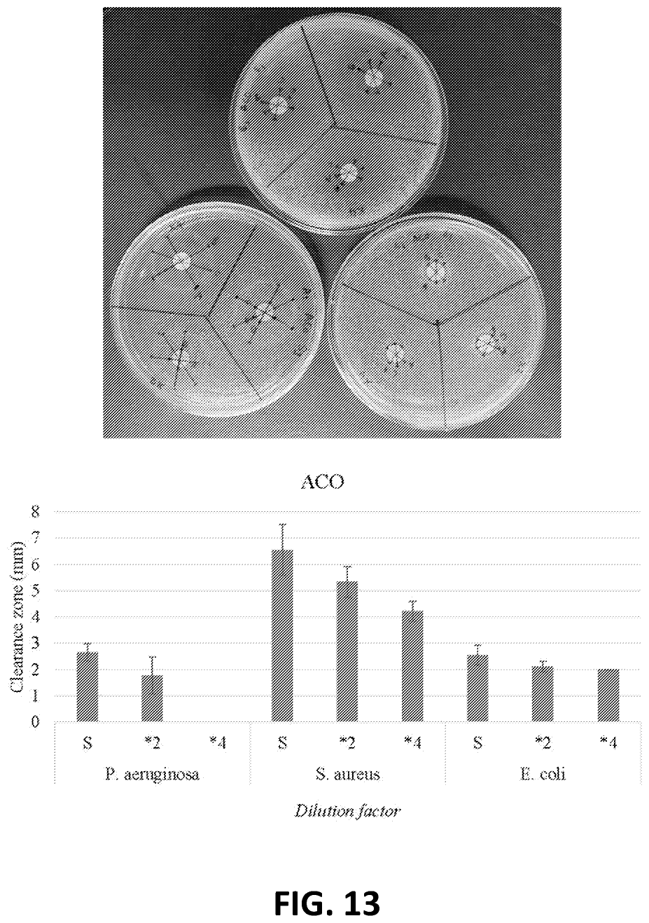

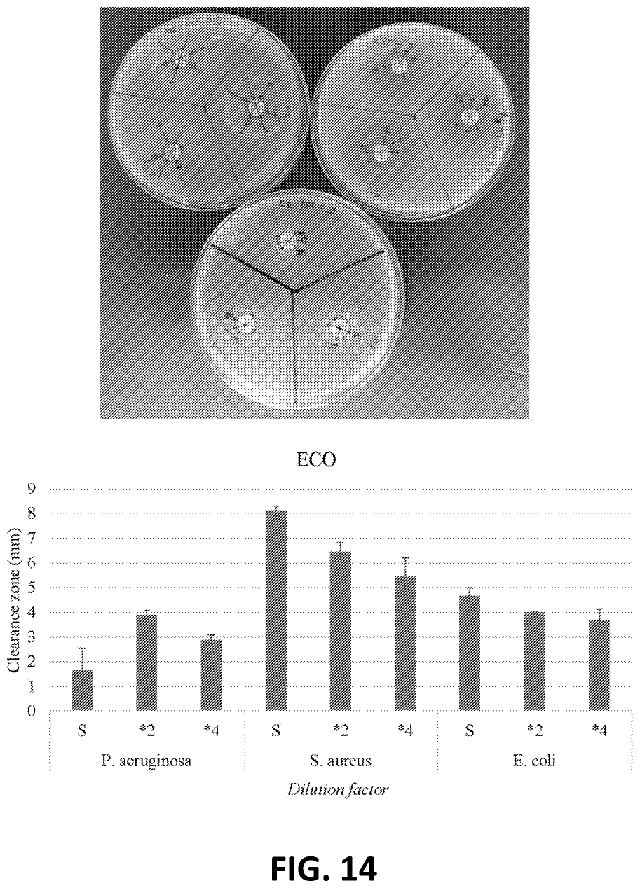

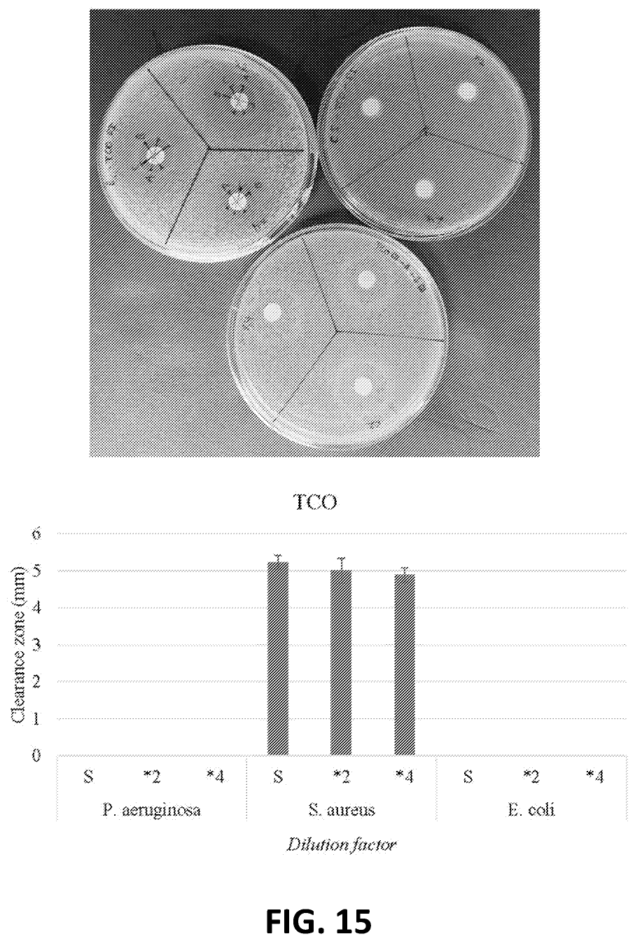

Next, the antibacterial activity of the organoantimony(V) cyanoximate compounds constructed in accordance with the present disclosure was analyzed. The procedure utilized is shown in FIG. 12. The various organoantimony(V) cyanoximate compounds were deposited in solid state, powder form on paper disks and tested against the pathogens P. aeruginosa, S. aureus, and E. coli. The results of these experiments are shown in FIGS. 13-15.

### Example 6

Disk diffusion assays: No inhibition of Cryptococcus neoformans or Candida albicans for the following organoantimony(V) cyanoximate compounds at any concentration (S, ×2, ×4): H(ACO), H(ECO), H(MCO).

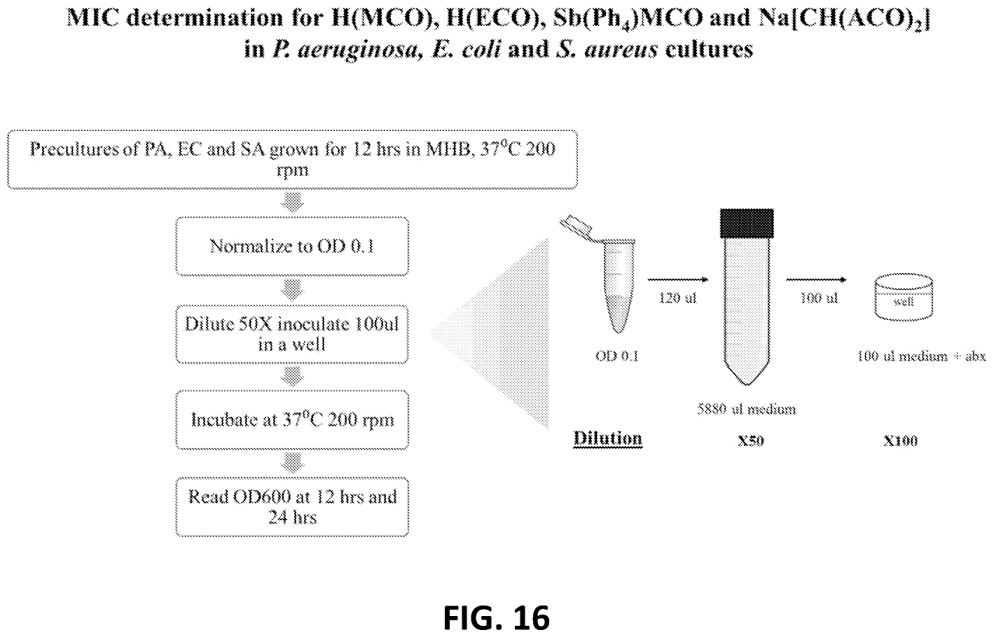

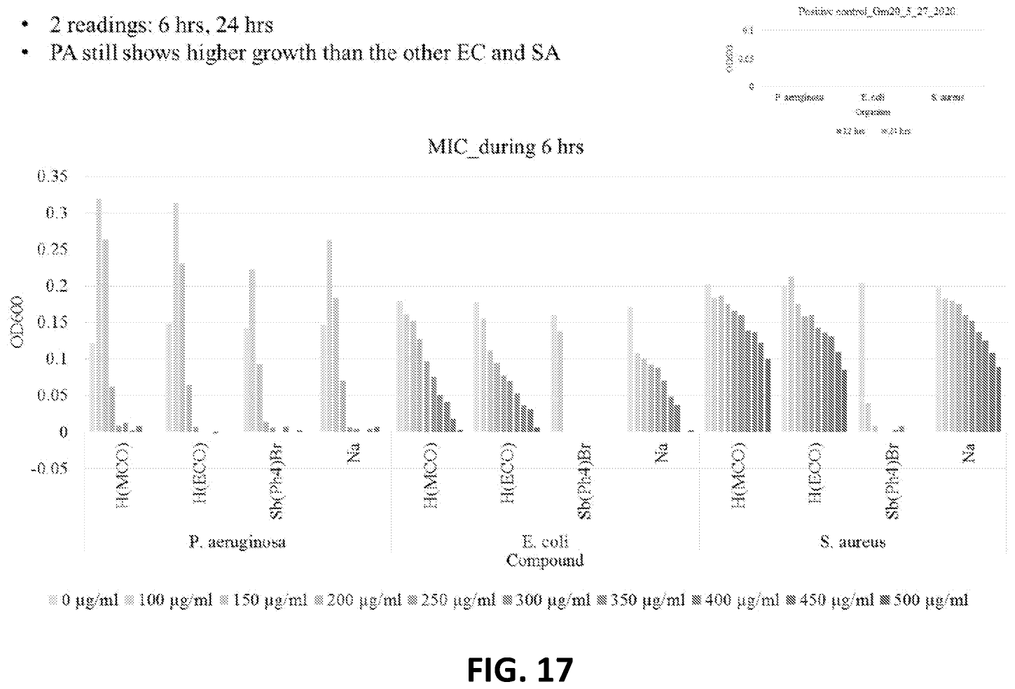

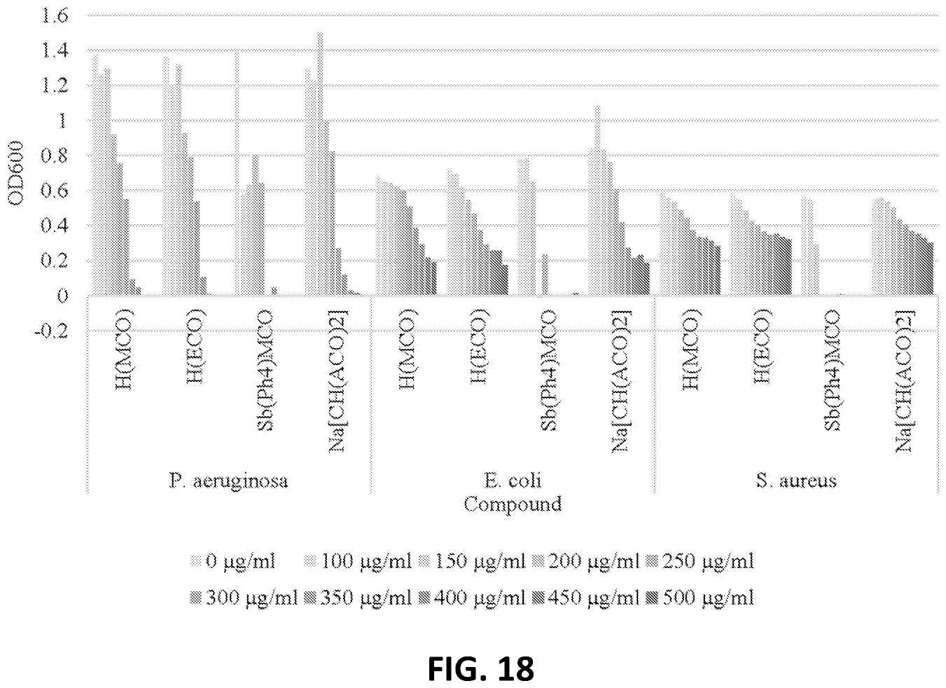

MIC assays: The MIC assay protocol utilized for determination of the effects of various organoantimony(V) cyanoximate compounds in various bacteria is shown in FIG. 16. Each solubilized in DMSO at 5 mg/ml, starting concentration is 100 μg/ml. Each compound was tested with each organism in three independent experiments, and the results are shown in FIGS. 17-18. Error bars represent standard error of the mean (if none, the values were identical in each experiment). Untested compounds have no bar. Bars reaching 100 μg/ml were not considered to have antifungal activity.

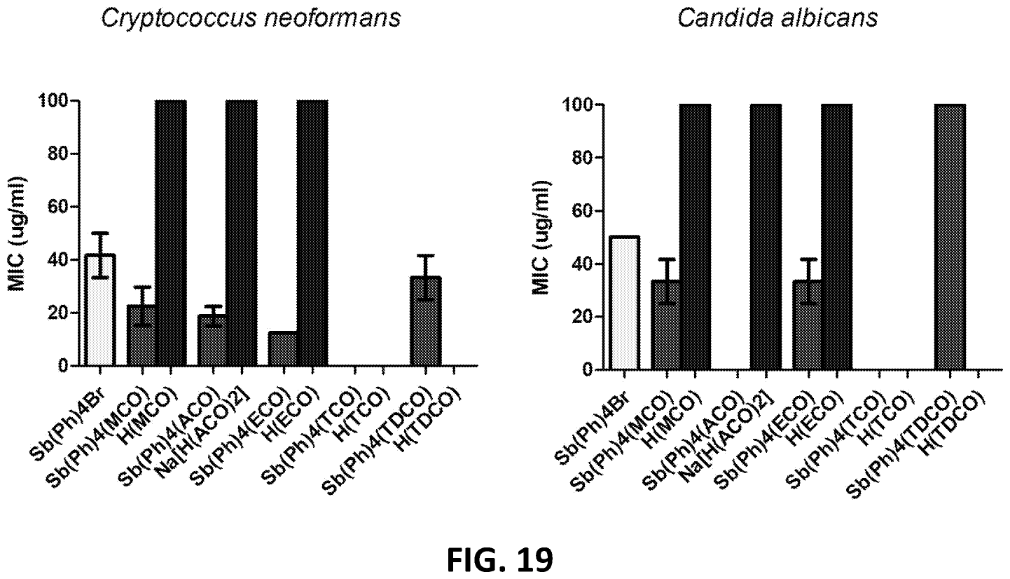

As shown in FIG. 19, several compounds tested had antifungal activity (i.e., inhibited growth). One compound—Sb(Ph)₄(MCO)—had fungicidal activity (killed C. neoformans) at a concentration of 100 μg/ml. MIC for this compound with C. neoformans was 22.5 μg/ml.

### Example 7

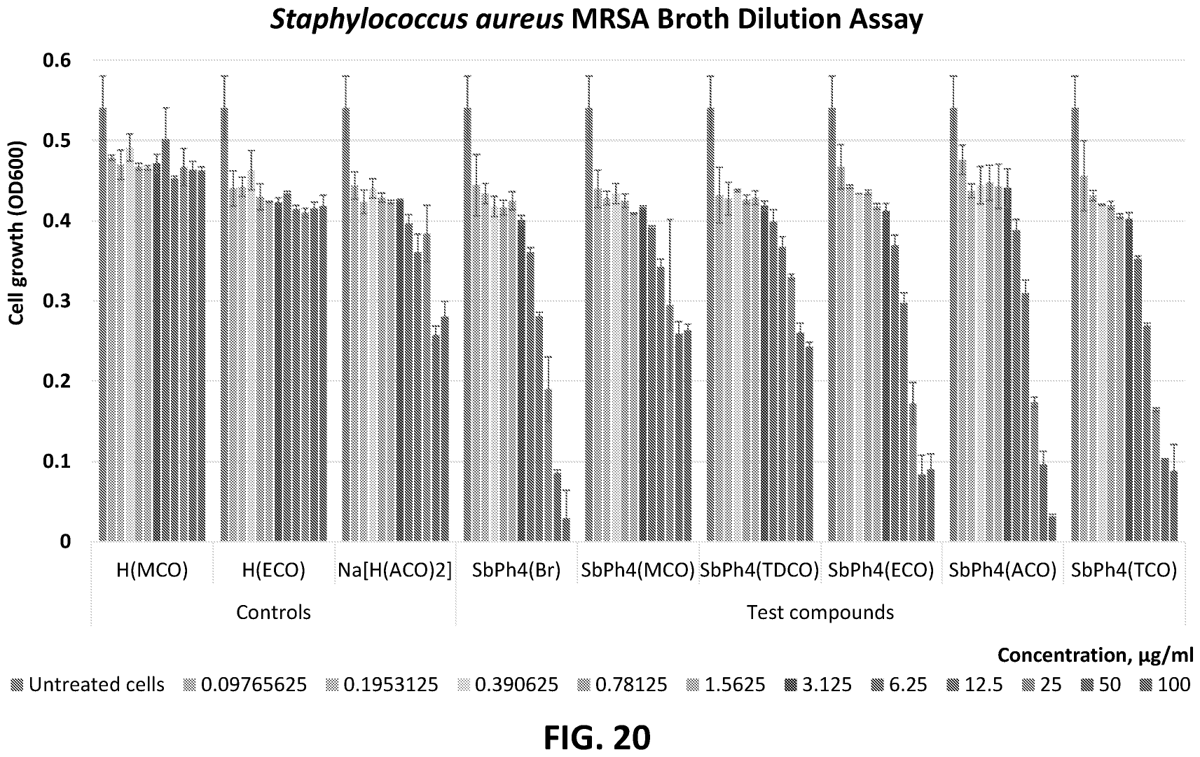

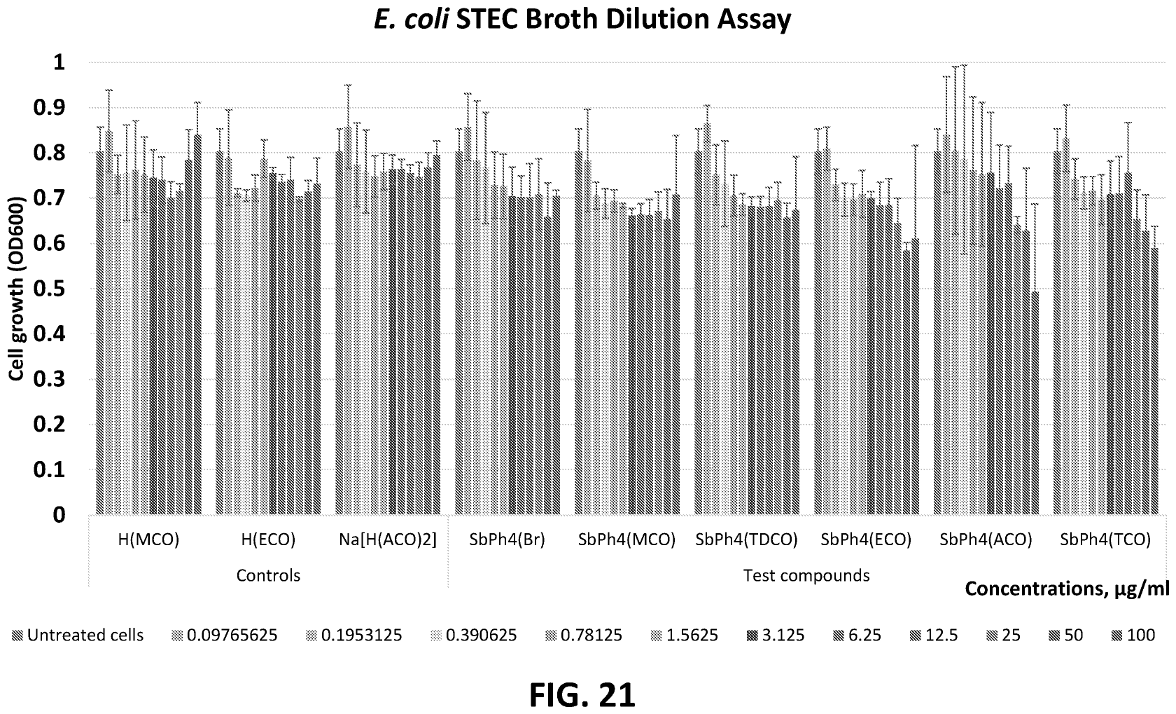

As shown in FIGS. 20-21, compounds containing Sb display higher bactericidal activity against Gram positive Staphylococcus aureus (FIG. 20) than Gram negative E. coli (FIG. 21). The Sb(V) cyanoximate compounds Sb(Ph4)Br, Sb(Ph4)ECO, Sb(Ph4)ACO, and Sb(Ph4)TCO showed significant bactericidal activity against multi-drug resistant strain of Gram positive S. aureus (FIG. 20), with Sb(Ph4)Br and Sb(Ph4)ACO showing MIC (minimum inhibitory concentrations) of 100 μg/ml. In addition, 25-40% reduction of growth was also observed for Sb(Ph4)Br, Sb(Ph4)ECO, Sb(Ph4)ACO, and Sb(Ph4)TCO against Gram negative E. coli (FIG. 21).

Thus, in accordance with the present disclosure, there have been provided compounds, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure,

1. A composition, comprising:
at least one organoantimony(V) cyanoximate compound of the formula:
SbR₄₋ₙL_{y},
wherein R comprises at least one of an aliphatic or an aromatic group, and L is a cyanoximate group;
wherein when n is 0, L is 1; when n is 1, L is 2; when n is 2, L is 3; and when n is 3, L is 4.

2. The composition of claim 1, wherein a cyanoxime molecule from which the organoantimony(V) cyanoximate compound is formed is selected from one of the following structures:

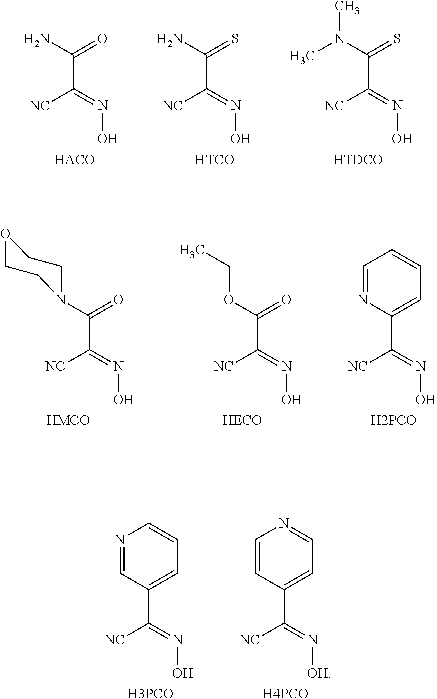

3. The composition of claim 1, wherein a cyanoxime molecule from which the organoantimony(V) cyanoximate compound is formed is selected from one of the following structures:

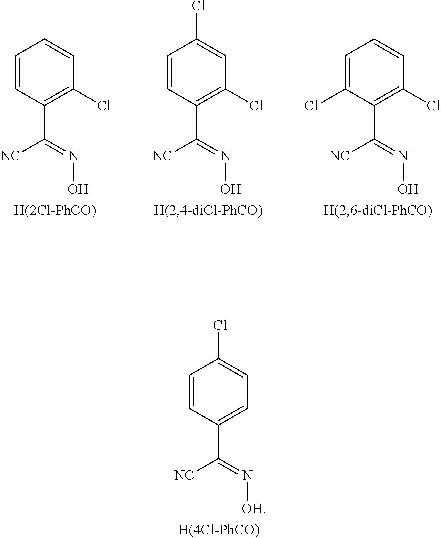

4. The composition of claim 1, wherein the organoantimony(V) cyanoximate compound is further defined as having one of the following structures: 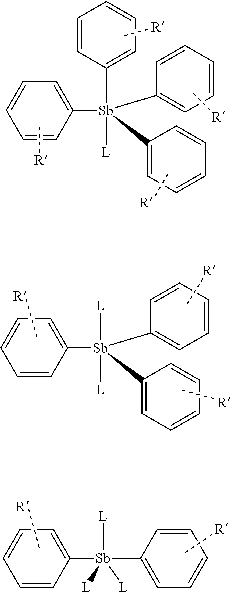
wherein each R′ comprises an aromatic group.

5. The composition of claim 4, wherein each R′ is a phenyl group.

6. The composition of claim 4, wherein each R′ aromatic group further comprises at least one of a halogen, alkyl, alkoxy, nitro-, or sulfo-group.

7. The composition of claim 1, wherein the organoantimony(V) cyanoximate compound is further defined as having one of the following structures: 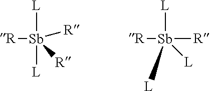
wherein each R″ comprises an alkyl chain and/or a cyclic alkyl group.

8. The composition of claim 7, wherein each R″ is a methyl group.

9. The composition of claim 1, wherein the organoantimony(V) cyanoximate compound is further defined as having the structure:

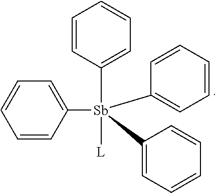

10. The composition of claim 1, wherein the organoantimony(V) cyanoximate compound is further defined as having the structure:

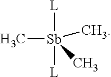

11. The composition of claim 1, wherein the at least one organoantimony(V) cyanoximate compound has antimicrobial activity.

12. The composition of claim 11, wherein the antimicrobial activity comprises antibacterial activity.

13. The composition of claim 11, wherein the antimicrobial activity comprises antifungal activity.

14. A composition, comprising:
an adhesive; and
at least one of the compositions of claim 1, wherein the at least one composition is incorporated within the adhesive.

15. A pharmaceutical composition, comprising:
at least one of the compositions of claim 1; and
a pharmaceutically-acceptable excipient or carrier.

16. A method of synthesizing at least one organoantimony(V) cyanoximate compound, the method comprising the steps of:
combining an organoantimony(V) compound with a cyanoxime to form a mixture; and
incubating the mixture under conditions whereby a metathesis reaction occurs and an organoantimony(V) cyanoximate compound is formed.

17. The method of claim 16, wherein the cyanoxime is selected from one of the following structures:

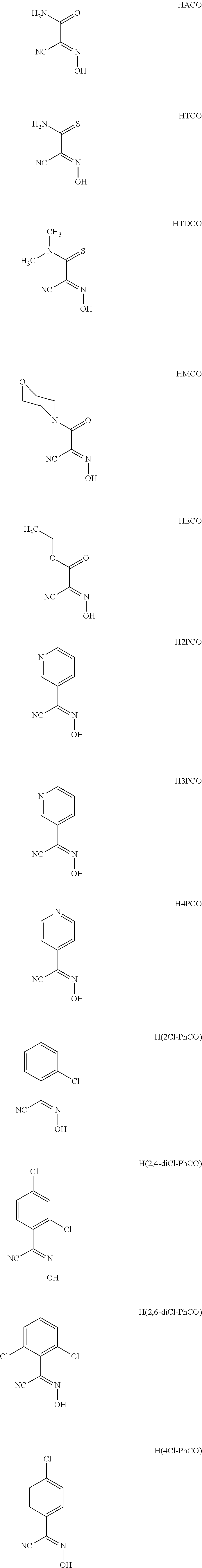

18. An assembly, comprising:
a device having at least one surface; and
the composition of claim 1 incorporated within the device and/or applied to at least a portion of the at least one surface of the device.

19. A method of reducing, inhibiting, and/or substantially preventing microbial growth on a device, the method comprising the step of:
applying the composition of claim 1 to at least a portion of a surface of the device; and/or
incorporating the composition within at least a portion of the device.

20. A method, comprising the step of:
administering an effective amount of the pharmaceutical composition of claim 15 to a patient in need thereof.
